(19) United States
(12) Patent Application Publication
(10) Pub. No.: US 2026/0132365 A1
(43) Pub. Date: May 14, 2026

(54) SINGLE-USE BIOREACTOR AND METHODS FOR USING THE SAME

(71) Applicant: Sartorius Stedim North America Inc., Bohemia, NY (US)

(72) Inventors: Michael A. Zumbrum, New Oxford, PA (US); William Kimmick, Mechanicsburg, PA (US); Kevin Perdue, Havre de Grace, MD (US)

(73) Assignee: Sartorius Stedim North America Inc., Bohemia, NY (US)

(21) Appl. No.: 19/385,654

(22) Filed: Nov. 11, 2025

Related U.S. Application Data
(60) Provisional application No. 63/719,796, filed on Nov. 13, 2024.

(51) Int. Cl. C12M 1/12; C12M 1/00; C12M 1/34

(52) U.S. Cl. CPC C12M 25/02; C12M 23/28; C12M 29/10; C12M 29/14; C12M 37/04; C12M 41/44

(57) ABSTRACT

A bioreactor includes a first shell a first outer membrane and the first growth membrane. The first shell includes a first sidewall and a first end wall. The first sidewall defines a first chamber and has a first open end and a second open end that is opposite the first open end. The first end wall extends across the first open end of the first sidewall. The second shell includes a second sidewall that defines a second chamber. The first outer membrane is secured to the first end wall and is configured to substantially seal the first chamber at the first end wall. The first growth membrane is disposed in the second chamber. The first growth membrane is microporous and is configured to allow liquid media to pass through while retaining cells with in the second chamber.

### CROSS-REFERENCE TO RELATED APPLICATION

This application claims benefit of, and priority to, U.S. Provisional Patent Application Ser. No. 63/719,796, filed Nov. 13, 2024, the entire contents of which are hereby incorporated by reference.

### BACKGROUND

### 1. Technical Field

The present disclosure relates to devices, systems, and methods for growing cells and, more specifically, to single-use bioreactors and methods for using the same to grow cells.

### 2. Discussion of Related Art

Cell expansion can be completed in shaker flasks, T-flasks, and other closed devices. These devices have limited ability to control gas diffusion and promote high density cell cultures. These devices can be difficult to use a method of continuous or semi-continuous perfusion to maximize cell growth.

### SUMMARY

This disclosure relates generally to a device for maximizing cell growth in a bioreactor system. The bioreactors disclosed herein may be single-use bioreactors having one or more growth chambers. This disclosure also relates to systems and methods for using the disclosed bioreactors in closed system processing. The disclosed bioreactor systems may be used with static media, perfused media, or recirculated media.

In an aspect of the present disclosure, a bioreactor includes a first shell, a second shell, a first membrane, and a second membrane. The first shell includes a first sidewall and a first end wall. The first sidewall defines a first chamber that has a first and the second end wall closes the first end. The second shell includes a second sidewall. The second sidewall defines a second chamber that has a second open end opposite the first end. The first membrane is secured across the second open end. The first membrane substantially seals the second chamber at the second open end. The second membrane is disposed between the first chamber and the second chamber. The second membrane is microporous and is configured to allow liquid media to pass through while retaining cells within the second chamber.

In aspects, the bioreactor reactor includes a lattice structure secured second shell. The lattice structure may include pads with the first membrane secured to the pads. The pads may be adhered to the first membrane or may be bonded to the first membrane.

In some aspects, the second membrane is a track etched membrane that defines a plurality of pores. Each pore may have a diameter of less than or equal to 50 micrometers. The second membrane may be formed of a porous hydrophilic material. The first membrane may include a semi-permeable layer of silicone. The semi-permeable layer of silicone may be configured to allow oxygen and carbon dioxide to permeate therethrough while preventing liquid from passing therethrough. The first membrane may include a non-woven fabric layer. The first membrane may define a plurality of pores with each pore having a diameter of less than or equal to 2 micrometers.

In some aspects, the second sidewall defines a first opening that is configured to allow for aseptic addition of media. The bioreactor may include ⅓ shell having ⅓ sidewall with the third sidewall defining a space below the first membrane. The bioreactor may be configured to be used with a static perfused or recirculated growth media.

In another aspect of the present disclosure, a bioreactor system includes a bioreactor as detailed herein with the first sidewall defining an outlet in the second sidewall defining an inlet. The bioreactor system also includes a pump that is configured to flow fluid into the inlet and the second sidewall such that the liquid flows into the second chamber and into the first chamber before discharging through the outlet defined in the first sidewall.

In aspects, the pump may be configured to recirculate liquid that flows from the outlet in the first sidewall back into the inlet defined in the second sidewall.

In another aspect of the present disclosure, a bioreactor includes a first sidewall, a first membrane, a second membrane, and a lattice structure. The first sidewall defines the growth chamber that is disposed between a first end and a second end defined by the first sidewall. The first membrane is disposed over and substantially sealing the first end. The second membrane is disposed over the second and is configured to retain cells within the growth chamber. The lattice structure is secured to the first sidewall. The first membrane is secured to the lattice structure.

In some aspects, the lattice structure is disposed within the growth chamber. The lattice structure may include pads. A top surface of the first membrane may be secured to the pads of the lattice structure. The first sidewall may include fee that extend below the first membrane to support the first membrane above a support surface.

In certain aspects, the first sidewall may define a port therethrough. The port may provide direct fluid communication into and out of the growth chamber. The first membrane may include a top surface and a bottom surface opposite the top surface. The top surface of the first membrane may be exposed to the growth chamber. The bottom surface of the first membrane may be completely unsupported.

In another aspect of the present disclosure, a method of expanding cells includes initiating flow of growth media into a bioreactor such that the growth media flows into a growth chamber of the bioreactor. The growth chamber may be defined by the first sidewall of the bioreactor, a first membrane that substantially seals a bottom of the growth chamber, and a second membrane that separates the growth chamber from the second chamber. The method may also include feeding cells to be expanded into the growth chamber. The method further includes controlling flow of growth media into the growth chamber of the bioreactor after feeding cells into the growth chamber such that the spent growth media flows through the second membrane into the second chamber before being discharged from the bioreactor after passing through the second chamber.

In another aspect of the present disclosure, a bioreactor includes a first shell, a second shell, a first outer membrane, and the first growth membrane. The first shell includes a first sidewall and a first end wall. The first sidewall defines a first chamber and has a first open end and a second open end that is opposite the first open end. The first end wall extends across the first open end of the first sidewall. The second shell includes a second sidewall that defines a second chamber. The first outer membrane is secured to the first end wall and is configured to substantially seal the first chamber at the first end wall. The first growth membrane is disposed in the second chamber. The first growth membrane is microporous and is configured to allow liquid media to pass through while retaining cells with in the second chamber.

In aspects, the bioreactor includes a first divider that separates the first chamber from the second chamber. The first growth member may be secured to the first. The first divider may be monolithically formed with the first shell the first divider may include a lattice the first growth membrane may be secured to the law of the first divider. The first divider may be at least one of a fabric, a screen, a lattice, or a porous membrane. The first divider may be disposed within this the first growth membrane.

In some aspects, the first growth membrane may be a track attached membrane that defines a plurality of pores with each for having a diameter of less than or equal to 50 μm. The first growth membrane may be formed of a porous hydrophilic material. The first outer membrane may include a semi-permeable silicone.

In particular aspects, the semi-permeable layer of silicone may be configured to allow oxygen and/or carbon dioxide to permeate therethrough while preventing liquid from passing therethrough. The first outer membrane may include a nonwoven fabric layer. The first outer membrane may define a plurality of pores of less than or equal to 2 micrometers.

In certain aspects, the second sidewall defines a first opening and the first opening is configured to allow for aseptic addition of media. The second shell may include a second end wall opposite the first divider. The second chamber may be defined by the first divider, the second end wall, and the second shell. The bioreactor may include a second outer membrane that is secured to the first end wall. The second outer membrane may be semi-permeable and configured to substantially seal the second chamber at the second end wall.

In aspects, the bioreactor includes a third shell, a second outer membrane, and a second growth membrane. The third shell may include a third sidewall and the second end wall. The third sidewall may define a third chamber and have a third open end and the fourth open end that is opposite the third open end. The second end wall may extend across the fourth open end of the second sidewall. The second outer membrane may be secured to the second end wall. The second outer membrane may be semi-permeable and configured to allow oxygen and/or carbon dioxide to permeate therethrough while preventing liquid from passing therethrough. The second growth membrane may be disposed in the second chamber. The second growth membrane may be microporous and configured to allow liquid media to pass through while retaining cells in within the second chamber. The bioreactor may include a second divider that separates the second chamber from the third chamber. The second divider may extend across the third open end. The second growth membrane may be secured to the second divider.

In some aspects, the second divider is monolithically formed with the third shell. The bioreactor may be configured to be used with a static, perfused, or recirculated growth media.

In another aspect of the present disclosure, a bioreactor system includes any bioreactor described herein and a pump. The first sidewall of the bioreactor may define an inlet and the third sidewall of the bioreactor may define an outlet. The pump is configured to flow liquid into the inlet and the first sidewall such that the liquid flows into the first chamber, through the second chamber, and into the third chamber before discharging through the outlet defined in the third sidewall.

In aspects, the pump is configured to recirculate liquid that flows from the outlet in the third sidewall back into the inlet defined in the first sidewall.

In another aspect of the present disclosure, a bioreactor includes a first shell, a second shell, ⅓ shell, a first divider, a first outer membrane, a second divider, second outer membrane, and a first growth membrane. The first shell includes a first sidewall and a first end wall. The first sidewall defines a first chamber and has a first open end and a second open end that is opposite to the first open end. The first end wall and asked dens across the first open end of the first sidewall. The second shell includes a second sidewall that defines a second chamber. This third shell includes a third sidewall and a second end wall. The third sidewall defines a third chamber and has a third open end and a fourth open end that is opposite to the first open end. The second end wall extends across the fourth open end of the second sidewall. The second divider separates the second chamber from the third chamber. The second divider extends across the third open end. The second outer membrane is secured to the second end wall. The second outer membrane is semi-permeable and is configured to allow oxygen and/or carbon dioxide to permeate therethrough wall preventing liquid from passing therethrough. The first growth membrane is secured to the first divider and is disposed in the second chamber. The first growth membrane is microporous and is configured to allow liquid media to pass through while retaining cells within the second chamber.

In aspects, the first sidewall, the second sidewall, and the third sidewall each define an opening. Each opening may be capable of allowing sampling of, feeding a fluid into, or discharge of fluid from a chamber defined by the respective sidewall.

In another aspect of the present disclosure, a bioreactor includes a first shell, the second shell, a first divider, a first outer membrane, and a first growth membrane. The first shell includes a first sidewall and a first end wall. The first sidewall define the first chamber and has first open end and a second open end that is opposite the first open end. The first end wall extends across the first open end of the first sidewall. The first end wall is formed of a first lattice structure. The second shell includes a second sidewall that defines a second chamber. The first divider separates the first chamber from the second chamber and is formed of a second lattice structure. The first outer membrane may be secured to the first lattice structure of the first end wall. The first outer membrane is configured to substantially seal the first chamber at the first end wall. The first growth membrane is secured to the second lattice structure of the first divider and is disposed in the second chamber. The first growth membrane is configured to allow liquid media to pass through while retaining cells within the second chamber. The first divider may be monolithically formed with the first shell.

In another aspect of the present disclosure, a method of expanding cells includes initiating flow of growth media into a bioreactor such that the growth media flows into a first chamber of the bioreactor, through a first divider of the bioreactor, and into a growth chamber of the bioreactor. The first divider may be disposed between the first chamber and the growth chamber. The method also includes feeding cells to be expanded into the growth chamber. The method further includes controlling flow of growth media into the bioreactor after feeding cells into the growth chamber such that spent growth media is discharged from the bioreactor after passing through the growth chamber.

In aspects, controlling flow of the growth media includes recirculating a portion of discharge growth media from the bioreactor into the first chamber. Recirculating discharge growth media may include recirculating discharge media from the growth chamber into the first chamber. Initiating flow of the growth media may include the growth media flowing through a growth membrane disposed within the growth chamber and secured to the first divider such that the growth membrane retaining cells to be expanded within the growth chamber. The growth media flowing through the growth membrane may include the growth membrane being formed of a track attached hydrophilic membrane. Initiating flow of the growth media may include the growth media flowing from the growth chamber through a second divider into ⅓ chamber. The second divider may be disposed between the growth chamber and the third chamber.

In some aspects, initiating flow of the growth media may include the growth media flowing through a growth membrane that is disposed within the growth chamber and secured to the second divider such that the growth membrane retains cells to be expanded within the growth chamber. Maintaining flow of the growth media may include recirculating discharged growth media from the third chamber of the bioreactor into the first chamber of the bioreactor. Recirculating discharged growth media may include mixing fresh growth media with the discharge growth media.

In certain aspects, the method may include allowing oxygen to permeate through an outer membrane that substantially seals an end wall of the third chamber. The end wall of the third chamber may be opposite the second divider. Allowing oxygen to permeate the outer membrane may include the outer membrane being formed of a hydrophobic silicone. Allowing oxygen to permeate the outer membrane may include the outer membrane having a layer of nonwoven fabric supporting the hydrophobic silicone.

In particular aspects, the method includes monitoring a property within the growth chamber with a sensor disposed in a sidewall that defines the growth chamber. The property may be temperature, pH, and/or density. The method may include adjusting flow of growth media based on the monitored property within the growth chamber. The method may include a septic please sampling directly from the growth chamber via an opening defined in a sidewall of the growth chamber.

Further, to the extent consistent, any of the embodiments or aspects described herein may be used in conjunction with any or all of the other embodiments or aspects described herein.

### BRIEF DESCRIPTION OF THE DRAWINGS

Various aspects of the present disclosure are described hereinbelow with reference to the drawings, which are not necessarily drawn to scale, which are incorporated in and constitute a part of this specification, wherein:

FIG. 1 is a perspective view of a bioreactor provided in accordance with embodiments of the present disclosure;

FIG. 2 is a perspective cutaway view of the bioreactor of FIG. 1;

FIG. 3 is a top perspective view, with parts separated, of the bioreactor of FIG. 1;

FIG. 4 is a top perspective view, with parts separated, of another reactor system provided in accordance with the present disclosure having two chambers;

FIG. 5 is a top perspective view, with parts separated, of a static reactor system provided in accordance with the present disclosure having a single chamber;

FIG. 6 is a flowchart of a method of growing cells in a static bioreactor provided in accordance with the present disclosure;

FIG. 7 is a flowchart of a method of growing cells in a perfusion bioreactor provided in accordance with the present disclosure;

FIG. 8 is a flowchart of a method of growing cells in a recirculating bioreactor provided in accordance with the present disclosure;

FIG. 9 is a top perspective view, with parts separated, of a recirculating reactor system provided in accordance with the present disclosure with three chambers;

FIG. 10 is a perspective view of another bioreactor provided in accordance with embodiments of the present disclosure;

FIG. 10A is a cutaway perspective view of a portion of the bioreactor of FIG. 10;

FIG. 11 is a longitudinal cross-sectional view of the bioreactor of FIG. 10;

FIG. 12 is a perspective view of another bioreactor provided in accordance with embodiments of the present disclosure;

FIG. 13 is a longitudinal cross-sectional view of the bioreactor of FIG. 12;

FIG. 14 is a perspective view of another bioreactor provided in accordance with embodiments of the present disclosure;

FIG. 15 is a longitudinal cross-sectional view of the bioreactor of FIG. 14;

FIG. 16 is a perspective view of another bioreactor provided in accordance with embodiments of the present disclosure; and

FIG. 17 is a longitudinal cross-sectional view of the bioreactor of FIG. 16;

FIG. 18 is a perspective view of another bioreactor provided in accordance with embodiments of the present disclosure;

FIG. 19 is an exploded view, with parts separated of the bioreactor of FIG. 18;

FIG. 20 is a longitudinal cross-sectional view of the bioreactor of FIG. 18;

FIG. 21 is a perspective view of another bioreactor provided in accordance with embodiments of the present disclosure; and

FIG. 22 is a longitudinal cross-sectional view of the bioreactor of FIG. 21.

### DETAILED DESCRIPTION

The present disclosure will now be described more fully hereinafter with reference to example embodiments thereof with reference to the drawings in which like reference numerals designate identical or corresponding elements in each of the several views. These example embodiments are described so that this disclosure will be thorough and complete, and will fully convey the scope of the disclosure to those skilled in the art. Features from one embodiment or aspect can be combined with features from any other embodiment or aspect in any appropriate combination. For example, any individual or collective features of method aspects or embodiments can be applied to apparatus, product, or component aspects or embodiments and vice versa. The disclosure may be embodied in many different forms and should not be construed as limited to the embodiments set forth herein; rather, these embodiments are provided so that this disclosure will satisfy applicable legal requirements. As used in the specification and the appended claims, the singular forms “a,” “an,” “the,” and the like include plural referents unless the context clearly dictates otherwise. In addition, while reference may be made herein to quantitative measures, values, geometric relationships, or the like, unless otherwise stated, any one or more if not all of these may be absolute or approximate to account for acceptable variations that may occur, such as those due to manufacturing or engineering tolerances or the like.

As used herein, the term “fluid communication” should be interpreted as allowing fluid to flow between two elements but with additional elements (tubes, lumens, chambers, etc.) between the identified elements. In contrast, as used herein, the term “direct fluid communication” should be interpreted as fluid flowing directly between the two elements without any elements therebetween. In addition, the phrase “configured to” describes a related element that must be made, suited by nature, character, or designed for a particular use, purpose, or situation. In contrast, the phrase “capable of” describes a related element that only needs to be suitable for a particular use, purpose, or situation.

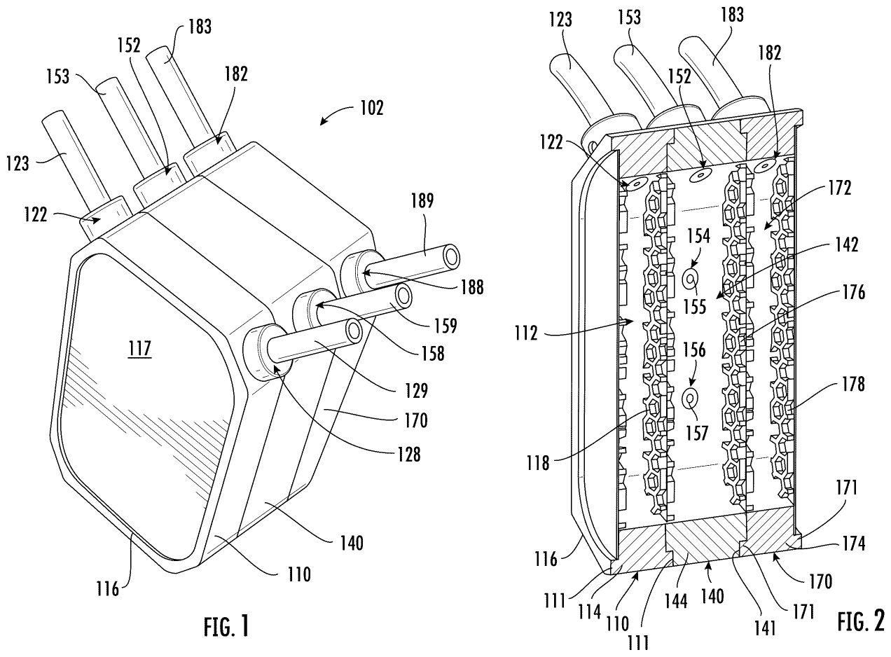

Referring now to FIGS. 1-3, a bioreactor 102 and a perfusion reactor system 100 are provided in accordance with the present disclosure. The bioreactor 102 includes three units or shells 110, 140, 170. The shells 110, 140, 170 are joined together to form three chambers 112, 142, 172 of the bioreactor 102. Each shell 110, 140, 170 includes sidewalls 114, 144, 174 that extend around the sides of the chambers 112, 142, 172 and are joined together with the sidewalls of the adjacent shells 110, 140, 170 to close the top, bottom, and sides of the chambers 112, 142, 172. The shells 110, 140, 170 may include protrusions 111, 171 and/or define recess 141 that cooperate with one another to align and secure the shells 110, 140, 170 together. The joints or seams between the shells 110, 140, 170 may be bonded, adhered, welded, or otherwise connected to one another to form the bioreactor 102.

The bioreactor 102 includes end walls 116, 178 and dividers 118, 176 that define the ends of the respective chambers 112, 142, 172. The end wall 116 is integrally formed with the sidewall 114 of the first shell 110 and the end wall 178 is integrally formed with the sidewall 174 of the third shell 170. In some embodiments, the end wall 116 and the end wall 178 may be monolithically formed with the respective first sidewall 114 or third sidewall 174. As used herein, the term monolithic means formed or composed of material without joints or seams. The end walls 116, 178 may include a lattice structure that is configured to support a membrane secured to the outside surface of the respective end wall 116, 178. The end walls 116, 178 may each include an outer membrane 117, 179 that is disposed on the outside surface of the respective end wall 116, 178. The outer membrane 117, 179 may be bonded, adhered, welded, or otherwise connected to the end wall 116, 178 to seal the bioreactor 102.

The outer membranes 117, 179 may be semi-permeable to substantially seal the respective end wall 116, 178 and may be configured to allow oxygen and carbon dioxide to permeate through the membrane 117, 179 while preventing liquid from passing through the outer membrane 117, 179. In embodiments, the outer membranes 117, 179 may be hydrophobic. In certain embodiments, one or both of the outer membranes 117, 179 may be semi-permeable to seal the respective end wall 116, 178 to control the rate of permeation of all fluids (including oxygen and carbon dioxide) through the membrane 117, 179. In particular embodiments, one outer membrane 117, 179 may be semi-permeable to allow permeation of oxygen and carbon dioxide therethrough and the other outer membrane 117, 179 prevents all fluids and gases from permeating therethrough. In certain embodiments, only a portion of the outer membrane 117, 179 is semi-permeable to allow permeation therethrough to limit evaporation. Outer membranes 117, 179 may be semi-permeable and contain pores less than 1 micrometer in size to allow oxygen and carbon dioxide to permeate through the outer membrane 117, 179 while preventing liquid from passing therethrough. In some embodiments, the outer membrane 117, 179 may be a hydrophobic membrane. In certain embodiments, one or both of the outer membranes 117, 179 may be considered semi-permeable. In some embodiments, a lattice structure of the end walls 116, 178 may support the outer membranes 117, 179. The outer membranes 117, 179 may be bonded, adhered, welded, or otherwise secured to the outer surface of the respective end wall 116, 178 such that the outer membrane 117, 179 substantially seals the respective end wall 116, 178. The lattice structure of the end walls 116, 178 may increase a surface area to secure the outer membrane 117, 179 thereto. The lattice structure of the end walls 116, 178 may provide structural support to the outer membrane 117, 179 that is secured thereto. In certain embodiments, the outer membranes 117, 179 may prevent evaporation of media from within the bioreactor 102. The outer membranes 117, 179 may be formed of an elastic membrane such as silicone. In certain embodiments, the outer membranes 117, 179 may be formed of a thin layer of silicone that is applied to a reinforcing material such as a woven fabric, a non-woven fabric, a screen, a porous membrane, or composites thereof. Applying silicone to a fabric may allow for the respective membrane to be thinner than a traditional silicone membrane without the fabric. For example, a silicone membrane with fabric reinforcement may have a silicone thickness in a range of 0.001 mm to 1 mm in thickness compared to a silicone membrane without fabric having a thickness in a range of 0.1 mm to 3 mm. The fabric may provide support to the membrane and prevent tearing of the thin layer of silicone. In some embodiments, the silicone may be partially or fully impregnated into the reinforcing material. In some embodiments, the membranes may be formed from more than 2 layers.

The divider 118 separates the first chamber 112 from the second chamber 142. The divider 118 may be integrally formed with the first sidewall 114 of the first shell 110 or the second sidewall 144 of the second shell 140. In some embodiments, the divider 118 is monolithically formed with the first sidewall 114 or the second sidewall 144. In certain embodiments, a portion of the divider 118 is integrally or monolithically formed with each of the first sidewall 114 and the second sidewall 144. The divider 176 separates the second chamber 142 from the third chamber 172. The divider 176 may be integrally formed with the second sidewall 144 of the second shell 140 or the third sidewall 174 of the third shell 170. In some embodiments, the divider 176 is monolithically formed with the second sidewall 144 or the third sidewall 174. In certain embodiments, a portion of the divider 176 is integrally or monolithically formed with each of the second sidewall 144 and the third sidewall 174. The dividers 118, 176 may include a lattice structure that is configured to support a growth membrane 130 thereon. The growth membranes 130a, 130b may be bonded, adhered, welded, or otherwise secured to the surfaces of the respective divider 118, 176 that face the growth chamber 142 such that the growth membranes 130a, 130b are disposed within the growth chamber 142. The lattice structure of the dividers 118, 176 may increase a surface area to secure the growth membrane 130a, 130b thereto. The lattice structure of the dividers 118, 176 may provide structural support to the growth membrane 130a, 130b that is secured thereto.

The growth membranes 130 are disposed on a side of the dividers 118, 176 that face the second chamber 142. In such embodiments, the second chamber 142 may be considered a growth chamber. The second chamber 142, and thus the growth membranes 130, may be configured to retain cells therein while allowing media and other fluids to flow through the dividers 118, 176. For example, media may flow from the first chamber 112, through the divider 118 into the second chamber 142, and through the divider 176 into the third chamber 172. The growth membranes 130a, 130b may be formed of a hydrophilic material. The growth membranes 130a, 130b may be formed of a microporous material that is configured to retain cells while allowing a media and other fluids to flow therethrough. The growth membranes 130a, 130 b may be configured to retain cells in a range of 1 micrometers to 50 micrometers. In some embodiments, the growth membranes 130a, 130b may be configured to block the passage of cells smaller than 1 micrometers or larger than 50 micrometers. The growth membranes 130a, 130b may be configured to allow a high flux of media therethrough. The growth membranes 130a, 130b may include a plurality of holes that pass through the growth membranes 130a, 130b to allow media to pass through. The plurality of holes may have a diameter in a range of 0.1 micrometer to 50 micrometers, e.g., 0.1, 0.5, 1, 2, 3, 4, 5, 6, 7, 8, 9, 10, 15, 20, 25, 30, 35, 40, 45, or 50 micrometers. One or more of the holes of the plurality of holes may pass directly through the respective growth membrane 130a, 130b or may be a tortuous path that extends through the respective growth membrane 130a, 130b. The plurality of holes may be created by a variety of means including, but not limited to, electron beam irradiation, laser drilling, microphase separation, or track etching. In embodiments, the growth membranes 130a, 130b may be formed of a polymer (polycarbonate, polysulfone, polyether sulfone, polyolefin, PVDF, cellulose acetate, cellulose nitrate, ePTFE, or polyester or polyamide) track-etched membrane filter with pores sized to retain a desired cell, e.g., pores in a range of 0.1 micrometer to 10 micrometers (for example, 1 micrometer). The track etched membrane may be made using polycarbonate, polyester, or polyamide. In some embodiments, the membranes may be formed of polyimide, (e.g., Kapton®) with a plurality of holes drilled therethrough. Specifically, Kapton® may vaporize when exposed to laser light at one or more frequencies. In addition, while Kapton® is known to be hydrophobic, it is possible to treat the surface such that Kapton® becomes hydrophilic. For example, the surface of the Kapton® may be surface treated with ultraviolet light to render the Kapton® hydrophilic. The ultraviolet light may be in a range of 100 nanometers to 400 nanometers. In certain embodiments, the ultraviolet light is ultraviolet-C light in a range of 100 nanometers to 280 nanometers, e.g., 100, 125, 150, 173, 175, 200, 225, 250, 275, or 280 nanometers. In certain embodiments, polyvinyl pyrrolidone (PVP) may be used as a coating material to render a hydrophobic membrane hydrophilic. Controlled pore size membranes may also be produced using additive manufacturing for polymeric, ceramic, and metallic materials.

The growth membranes 130a, 130b may include a support or reinforcement to support or secure the growth membrane 130a, 130b to the respective divider 118, 176. For example, a support or reinforcement may be on a side of the growth membrane 130a facing or away from the second chamber 142 such that the growth membrane 130a is secured to the divider 118 and remains attached when exposed to media flow from the first chamber 112 towards the third chamber 172. The support or reinforcement may be on a side of the growth membrane 130b that is away from the second chamber 142 to prevent the growth membrane 130b from passing through the divider 176. While the support or reinforcement of the growth membranes 130a, 130b may be the same because the requirements of the support or reinforcement is different based on the position of the respective growth membrane 130a, 130b each growth membrane 130a, 130b may have a different support or reinforcement. The support or reinforcement may be a stainless-steel mesh screen that is disposed on a side of the respective growth membrane 130a, 130b. Such a stainless-steel mesh screen may have openings or voids in a range of 50 micrometers to 1,500 micrometers such that flow of media through the respective growth membrane 130a, 130b is not significantly impeded by the reinforcement. In some embodiments, the reinforcement or support may be a non-woven fabric. Such a non-woven fabric may be formed of polyester, polyolefins, polyamide, polyethylene, polytetrafluoroethylene, or polyurethane fibers. In certain embodiments, Tyvek® available from Dupont® may be used as a support or reinforcement for the growth membranes 130a, 130b. The growth membranes 130a, 130b may be bonded, adhered, welded, or otherwise secured to the outer surface of the respective divider 118, 176 such that the growth membrane 130a, 130b substantially attaches to the respective divider 118, 176 and creates a sealed bioreactor.

In certain embodiments, the divider 118 and/or the divider 176 may be a reinforcing material to support the respective growth membrane 130a, 130b in the form of a fabric, a screen, a porous membrane, or combinations thereof. In such embodiments, the dividers 118, 176 may be formed separate from the shells. In some embodiments, the divider 118, 176 may be integrated with a respective growth membrane 130a, 130b. For example, the growth membrane 130a, 130b may be disposed on one side of the reinforcing material or may be disposed about the reinforcing material. In such embodiments, the growth membranes 130a, 130b may be considered self-supporting with the reinforcing material integrated therewith to separate the respective chamber from the growth chamber and to block the passage of cells from the growth chamber.

In some embodiments, the growth membranes 130a, 130b are formed with additive manufacturing techniques to include pores that are sized to prevent cells from passing through the respective membrane. The growth membranes 130a, 130b may be formed with uniform sized pores that pass linearly through the respective membrane or with pores that are formed with a tortuous path, or combinations of linearly and tortuous pores. In particular embodiments, the growth membranes 130a, 130b may be part of a respective one of the dividers 118, 176 such that the growth membrane 130a, 130b is monolithically formed with the respective divider 118, 176.

Each sidewall 114, 144, 174 may define one or more fittings or openings to allow fluid to flow into or out of the respective chamber 112, 142, 172. The openings may be used to sample a fluid within the respective chamber, provide ingredients into the respective chamber, perfusion of media, addition or removal of cells from the respective chamber, or other purpose for accessing the respective chamber. The first chamber 112 defined by the first shell 110 may define a first chamber inlet 122 and a first chamber outlet 128. The first chamber inlet 122 or the first chamber outlet 128 may include a tube 123, 129 or a septum disposed therein. When a septum is disposed in first chamber inlet 122 or the first chamber outlet 128, the septum may be punctured to access the first chamber 112. In certain embodiments, the septum may be resealable. In particular embodiments, the septum may be aseptically resealable. When a tube 123, 129 extends from the first chamber inlet 122 or the first chamber outlet 128, the tube 123, 129 may allow for fluid to flow into or out of the first chamber 112. The tubes 123, 129 may allow for sampling from the chamber 112. The tubes 123, 129 may include a connector to allow for aseptic fluid connection to the first chamber 112. The first chamber inlet 122 or the first chamber outlet 128 may allow for a vent or a pressure relief port to be in fluid communication with the first chamber 112. The second shell 140 and the third shell 170 may include respective inlets 152, 182 and outlets 158, 188 that are similar to the inlet 122 and the outlet 128 respectively and will not be discussed for reasons of brevity.

The second chamber 142 may include one or more ports 154, 156 disposed in the respective sidewall 144. The ports 154, 156 may each receive a sensor 155, 157 to measure one or more properties of a fluid within the second chamber 142. The sensors 155, 157 may measure a pH, a temperature, a density, cell density, oxygen concentration, cell viability, or other property of the fluid or cells. While the ports 154, 156 are only shown in the second chamber 142, the first chamber 112 or the third chamber 172 may include ports similar to the ports 154, 156.

The bioreactor 102 described above is a triple or three chamber bioreactor. As shown, the first chamber 112 is a media or inflow chamber, the second chamber 142 is the growth chamber, and the third chamber 172 is a media or outflow chamber with media flowing into the first chamber 112, through the second chamber 142, and into the third chamber 172 before exiting the bioreactor 102. It is contemplated that more than three shells can be stacked together to form a bioreactor having more than three chambers. In such embodiments, multiple growth chambers may be stacked adjacent to one another and/or one or more additional media chambers may be disposed between growth chambers. For example, a seven-chamber bioreactor may be stacked, in order, with a media chamber, two growth chambers, a media chamber, two growth chambers, and a media chamber. Alternatively, a seven-chamber bioreactor may be stacked in order, with a media chamber, a growth chamber, a media chamber, a growth chamber, a media chamber, a growth chamber, a media chamber. The number of chambers and type of chambers may be determined by a cell being grown or a recipe of media.

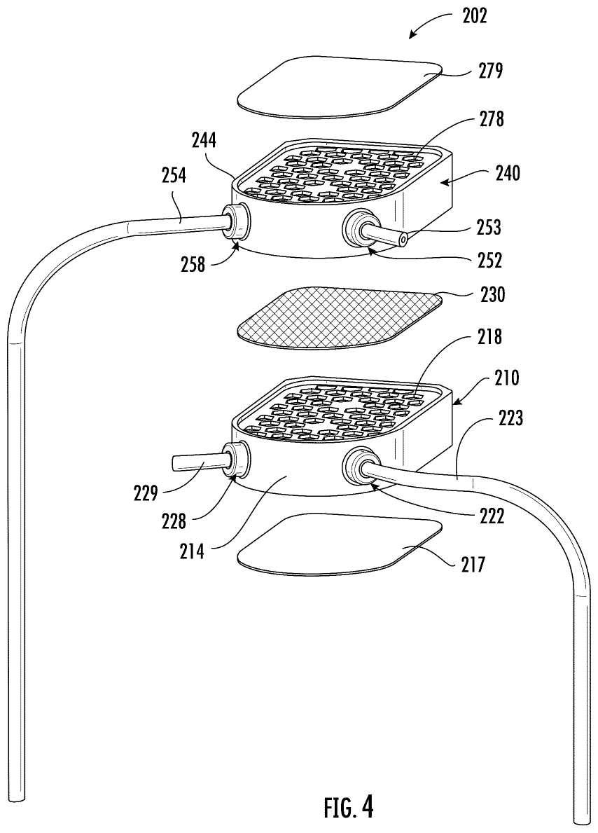

Referring now to FIG. 4, another bioreactor 202 is provided in accordance with embodiments of the present disclosure. The bioreactor 202 is similar to the bioreactor 102 detailed above with like elements labeled with a similar label with a leading “2” replacing the leading “1” of the label of the similar element of the bioreactor 102 with only the differences detailed herein for brevity.

The bioreactor 202 is a dual or two chamber bioreactor. The bioreactor 202 includes a first shell 210 and a second shell 240 that define a first chamber and a second chamber, respectively. As shown, the second chamber is a growth chamber and the first chamber is a media chamber. The bioreactor 202 includes outer membranes 217, 279 that seal or substantially seal the end walls of the bioreactor 202 and a divider 218 that separates the first chamber from the second chamber. The bioreactor 202 also includes a growth membrane 230 that is disposed on or formed with the divider 218 such that the growth membrane 230 is disposed in the second chamber. The divider 218 may be coasted with an adhesive to secure the growth membrane 230a thereto. For example, platinum catalyzed silicone adhesives or pressure sensitive acrylic adhesives may be used to secure the growth membrane 230a to the divider 218. Thermal heat staking may be used to bond the divider to the chamber. The bioreactor 202 may also be operated with the growth chamber defined by the first shell 210. This media may be pumped into the first chamber and retain the cells with the growth membrane 230 in the center.

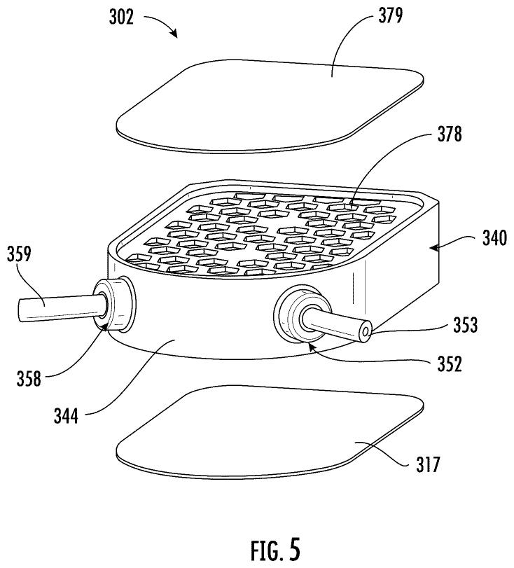

With reference to FIG. 5, another bioreactor 302 is provided in accordance with embodiments of the present disclosure. The bioreactor 302 is similar to the bioreactor 102 detailed above with like elements labeled with a similar label with a leading “3” replacing the leading “1” of the label of the similar element of the bioreactor 102 with only the differences detailed herein for brevity. The height of the growth chamber can be made as tall as necessary to maximize growth. Air above the growth liquid may improve cell growth. Rocking or other agitation in conjunction with or without air may improve cell growth.

The bioreactor 302 is a single or one chamber bioreactor. The bioreactor 302 includes a shell 340 that defines a growth chamber. The bioreactor 302 includes outer membranes 317, 379 that seal or substantially seal the end walls of the bioreactor 302. The bioreactor 302 may include one or more growth membranes on the internal sides of the end walls of the bioreactor 302 such that the growth membranes are disposed in the growth chamber. In some embodiments, the bioreactor 302 may be provided without a growth membrane. The bioreactor 302 may be used as a static chamber reactor as described below.

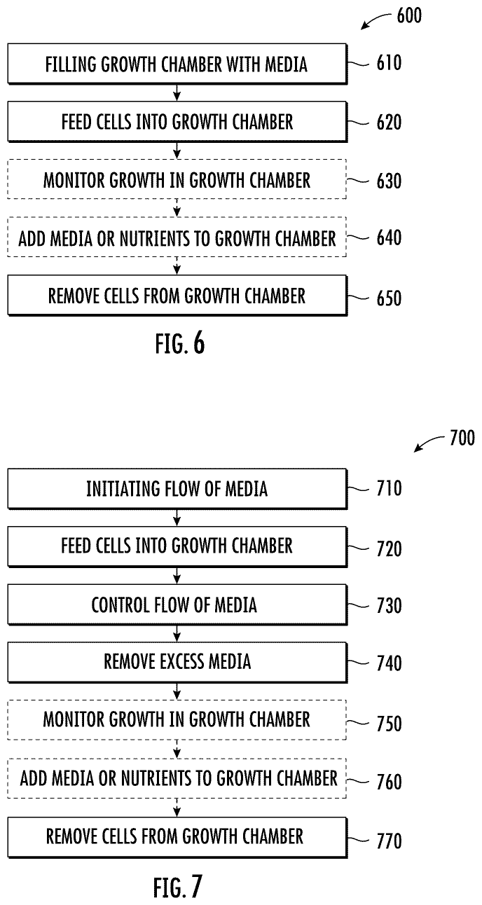

Referring now to FIG. 6, a method of expanding cells in a static bioreactor is described in accordance with embodiments of the present disclosure and is referred to generally as method 600 and is described with reference to bioreactor 202 of FIG. 4. The method 600 may utilize any of the bioreactors detailed herein, e.g., bioreactors 102, 202, or 302. The method 600 includes filling the growth chamber of the bioreactor 202 with growth media (Step 610). The growth media may be fed into the growth chamber via the inlet 222 such that the growth media fills the first chamber and the growth chamber. During filling, the bioreactor 202 may be oriented with the openings 222, 228 on a top side of the bioreactor 202 with a portion of the sidewall opposite the openings 222, 228 on a surface supporting the bioreactor 202. Once filled, the bioreactor 202 may be repositioned to a horizontal position with the end wall of the first chamber, and thus the outer membrane 217, facing downward. When the outer membrane 217 faces downward, the outer membrane 217 may fully seal the end wall such that no gases pass through the outer membrane 217. In some embodiments, the bioreactor 202 may be filled in the horizontal position, allowing liquid to displace the air in the chamber and exit through a sterile vent mean the outlet. In certain embodiments, the bioreactor may be filled in the horizontal position when sterile vent filters are used to allow gas to escape from the device when filling.

With the growth chamber filled or substantially filled with growth media, cells to be expanded are fed into the growth chamber of the bioreactor 202 via the inlet 252 (Step 620). The additional media chambers outside of the growth chamber may provide additional nutrients for the cells within the growth chamber while keeping the entire system closed. During the expansion of the cells, the outer membranes 217, 279 may allow for gases such as oxygen, carbon dioxide, and water vapor to transport into or out of the growth chamber. The outer membranes 217, 279 may be semi-permeable to allow for gases such as oxygen and carbon dioxide to permeate into and out of the growth chamber.

Once the cells are within the growth chamber, the growth of the cells or other variables within the growth chamber may be monitored with sensors disposed in the ports of the growth chamber (Step 630). In some embodiments, additional media, nutrients, or other additives may be added to the bioreactor 202 as a result of the monitored variables (Step 640). Adding media, nutrients, or other additives may increase a density of cell growth or enhance viability of the cells. During the growth, the cells may be sampled one or more times via one of the ports of the growth chamber. When the cells within the growth chamber reached a desired state, the cells are extracted or discharged from the growth chamber via the outlet 254 of the growth chamber (Step 650). The properties of the cells within the growth chamber may be determined by monitoring of the growth chamber, a passage of time, or other measurement. When the cells are removed from the bioreactor 202, the bioreactor 202 may be disposed of or otherwise recycled. In some embodiments, the growth media may be removed or recharged and additional cells grown within the growth chamber.

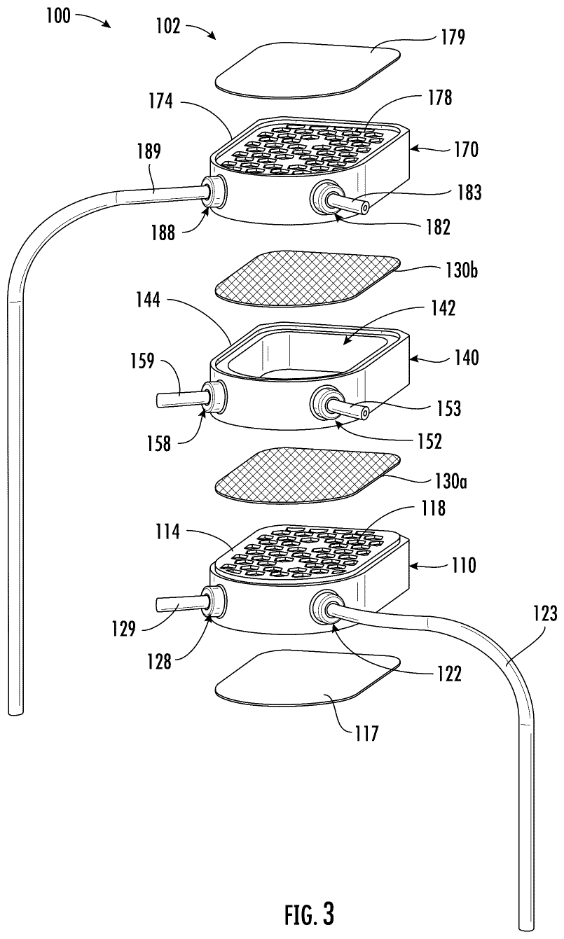

With reference to FIG. 7, a method of expanding cells in a perfusion bioreactor system is described in accordance with embodiments of the present disclosure and is referred to generally as method 700 and is described with reference to bioreactor system 100 of FIG. 3. The method 700 may utilize any of the multi-chamber bioreactors detailed herein, e.g., bioreactor 102, 202. The method 700 includes initiating flow of growth media into the first chamber of the bioreactor 102 (Step 710). The growth media may be fed into the first chamber via the tube 123 that is fluidly coupled to the first chamber via the first inlet 122. As the growth media is fed into the first chamber, the media fills the first chamber and passes through the divider 118 and into the growth chamber 142. The growth media may be fed directly into the growth chamber via the inlet 152 such that the growth media fills the growth chamber and flows through the divider 118 into the first chamber. During filling, the bioreactor 102 may be oriented with the openings 122, 128 on a top side of the bioreactor 102 with a portion of the sidewall opposite the openings 122, 128 on a surface supporting the bioreactor 102. Once filled, the bioreactor 102 may be repositioned to a horizontal position with the end wall of the first chamber, and thus the outer membrane 117, facing downward. When the outer membrane 117 faces downward, the outer membrane 117 may fully seal the end wall such that no gases pass through the outer membrane 117. In some embodiments, the bioreactor 102 may be filled in the horizontal position. The flow of growth media may be provided by a pump. The pump may be a peristaltic pump that receives the tube 123 therein to provide fluid to the bioreactor 102. The pump may be operated continuously or controlled to change flow rate depending upon the recipe or via feedback from sensors.

With the growth chamber filled or substantially filled with growth media, cells to be expanded are fed into the growth chamber of the bioreactor 102 via the inlet 152 (Step 720). After the cells are fed into the growth chamber, the flow of growth media into the bioreactor 102 is maintained (Step 730). Controlling the flow of growth media may continue to provide nutrients to the cells within the growth chamber. The flow of growth media may be provided via the tube 123 through the inlet 122. While the flow is maintained, excess growth media may be removed from the outlet 188 via the tube 189 (Step 740). The inlet 182 may include a pressure relief valve to prevent over pressurization within the bioreactor 102. The flow of growth media may flow from the first chamber, through the growth chamber, and into the third chamber before exiting the bioreactor 102. The growth media that is removed from the bioreactor 102 may be depleted before being removed. Depleted growth media may be media in which a substantial percentage of nutrients in the growth media is utilized by the cells. The continued flow of growth media may provide oxygen or other gasses dissolved in the growth media to the cells. As the growth media flows through the bioreactor 102, the growth membranes 130a, 130b allow the growth media to flow through the bioreactor 102 while preventing cells from exiting the growth chamber. During the expansion of the cells, the outer membranes 117, 179 may be semi-permeable to allow for gases such as oxygen to permeate into the growth chamber. The outer membranes 117, 179 may allow for gases such as carbon dioxide to permeate out of the growth chamber.

Once the cells are within the growth chamber, the growth of the cells or other variables within the growth chamber may be monitored with sensors disposed in the ports of the growth chamber (Step 750). In some embodiments, additional media, nutrients, or other additives may be added to the bioreactor 102 as a result of the monitored variables (Step 760). Adding media, nutrients, or other additives may increase a density of cell growth or enhance viability of the cells. The additional media, nutrients, or other additives may be added via openings 122, 152, 182. In certain embodiments, the flow of growth media is increased or decreased in view of the monitored variables. During the growth, the cells may be sampled one or more times via one of the openings of the growth chamber. When the cells within the growth chamber reached a desired density, the cells are extracted or discharged from the growth chamber via the opening 158 of the growth chamber (Step 770). The health of the cells within the growth chamber may be determined by monitoring of the growth chamber, a passage of time, or other measurement. When the cells are removed from the bioreactor 102, the bioreactor 102 may be disposed of or otherwise recycled. In some embodiments, the growth media may be removed or recharged and additional cells grown within the growth chamber.

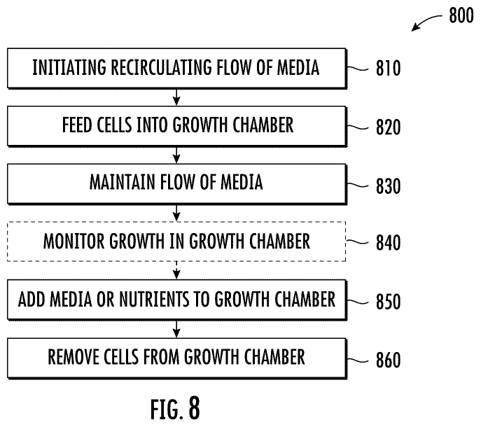

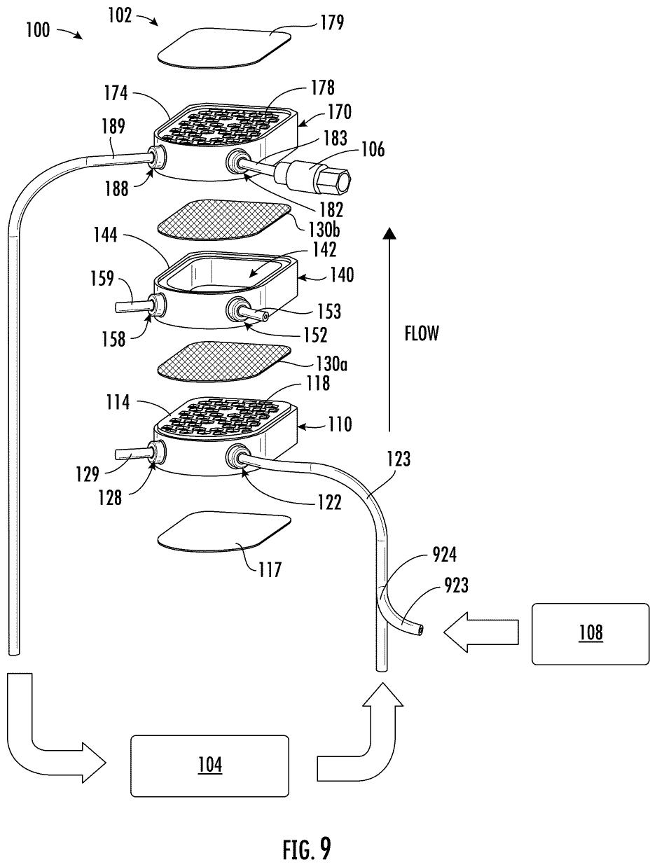

With reference to FIGS. 8 and 9, a method of expanding cells in a recirculation bioreactor system is described in accordance with embodiments of the present disclosure and is referred to generally as method 800 and is described with reference to bioreactor system 100 of FIG. 9. The method 800 may utilize any of the multi-chamber bioreactors detailed herein, e.g., bioreactor 102, 202. The bioreactor system 101 utilizes the bioreactor 102 but with a recirculation pump 104 disposed between the tube 189 and the tube 123 such that growth media that is removed from the third chamber may be recirculated back into the first chamber. The tube 123 or the tube 189 may include a filter. The tube 123 may include an inflow tube 923 to provide new growth media or other additives into the bioreactor 102. The inflow tube 923 may be connected via a one-way valve 924 to prevent backflow through the inflow tube 923. The inflow tube 923 may be in fluid communication with a growth media source.

The method 800 includes initiating recirculating flow of growth media into the first chamber of the bioreactor 102 (Step 810). The growth media may be fed into the first chamber via the tube 123 that is fluidly coupled to the first chamber via the first inlet 122. As the growth media is fed into the first chamber, the media fills the first chamber and passes through the divider 118 and into the growth chamber 142. When the flow of growth media is initiated, the growth media flows through the bioreactor 102 from the first chamber, through the growth chamber, and into the third chamber with excess fluid flowing through the opening 188 into a return tube 189. As the growth media flows through system 101, the growth media may be reoxygenated and/or filtered before being returned to the bioreactor 102. The opening 182 may include a pressure relief valve 106 to prevent over pressurization within the bioreactor 102. The flow of growth media may provide oxygen or other gasses dissolved in the growth media to the cells. As the growth media flows through the bioreactor 102, the growth membranes 130a, 130b allow the growth media to flow through the bioreactor 102 while preventing cells from exiting the growth chamber. During the expansion of the cells, the outer membranes 117, 179 may allow for gases such as oxygen to permeate into the growth chamber. The outer membranes 117, 179 may allow for gases such as carbon dioxide to permeate out of the growth chamber.

When the flow of growth media recirculating through the system 101, cells to be expanded are fed into the growth chamber of the bioreactor 102 vis the inlet 152 (Step 820). After the cells are fed into the growth chamber, the flow of growth media the bioreactor 102 is maintained (Step 830). Maintaining the flow of growth media may continue to provide nutrients to the cells within the growth chamber. The flow of growth media may be provided via the tube 123 through the inlet 122. In embodiments, the flow of growth media may be continuous, intermittent, varied in response to properties measured by sensors, or controlled by a growth recipe.

Once the cells are within the growth chamber, the growth of the cells or other variables within the growth chamber may be monitored with sensors disposed in the ports of the growth chamber (Step 840). In some embodiments, additional media, nutrients, or other additives may be added to the bioreactor 102 as a result of the monitored variables (Step 850). Adding media, nutrients, or other additives may increase a density of cell growth or enhance viability of the cells. The additional media, nutrients, or other additives may be added via openings 122, 152, 182. In embodiments, the tube 189 may be used. In certain embodiments, the flow of growth media is increased or decreased in view of the monitored variables. During the growth, the cells may be sampled one or more times via one of the openings of the growth chamber. When the cells within the growth chamber reached a desired density, the cells are extracted or discharged from the growth chamber via the opening 158 of the growth chamber (Step 860). The density of the cells within the growth chamber may be determined by monitoring of the growth chamber, a passage of time, or other measurement. When the cells are removed from the bioreactor 102, the bioreactor 102 may be disposed of or otherwise recycled. In some embodiments, the growth media may be removed or recharged and additional cells grown within the growth chamber.

Although the method steps are described in a specific order, it should be understood that other steps may be performed in between described steps, described steps may be adjusted so that they occur at slightly different times, or the described steps may occur in any order unless otherwise specified.

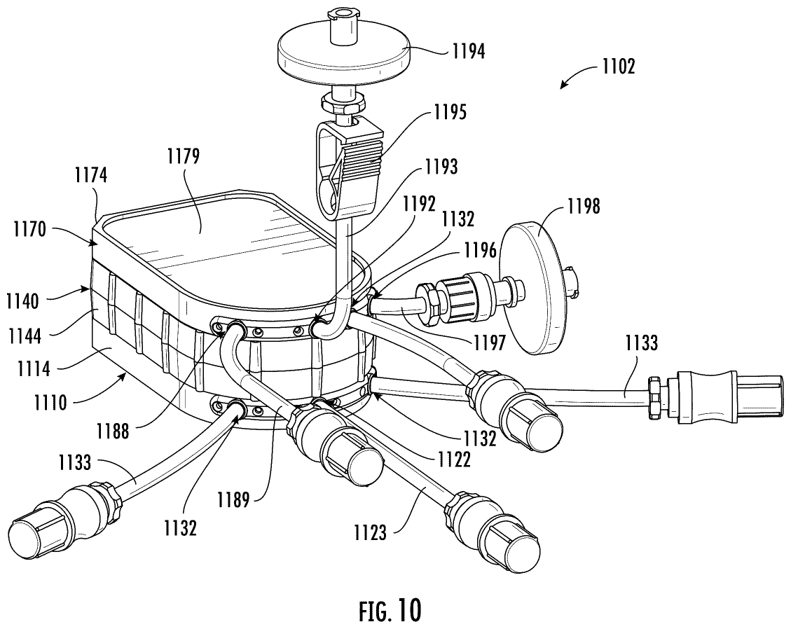

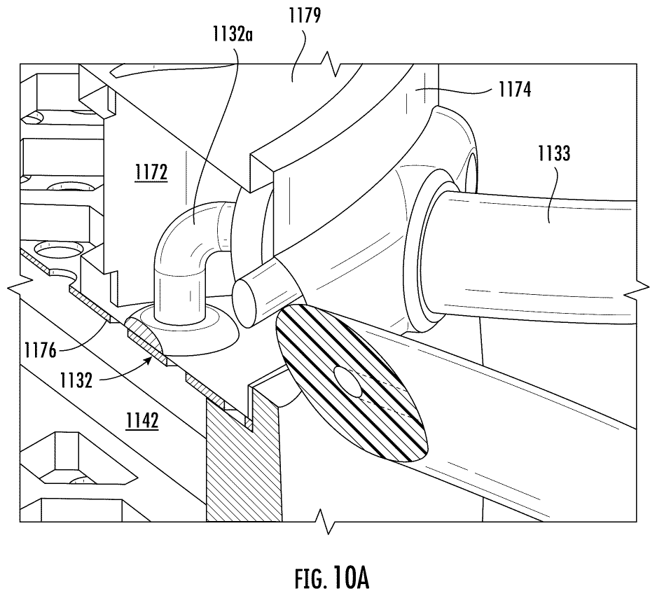

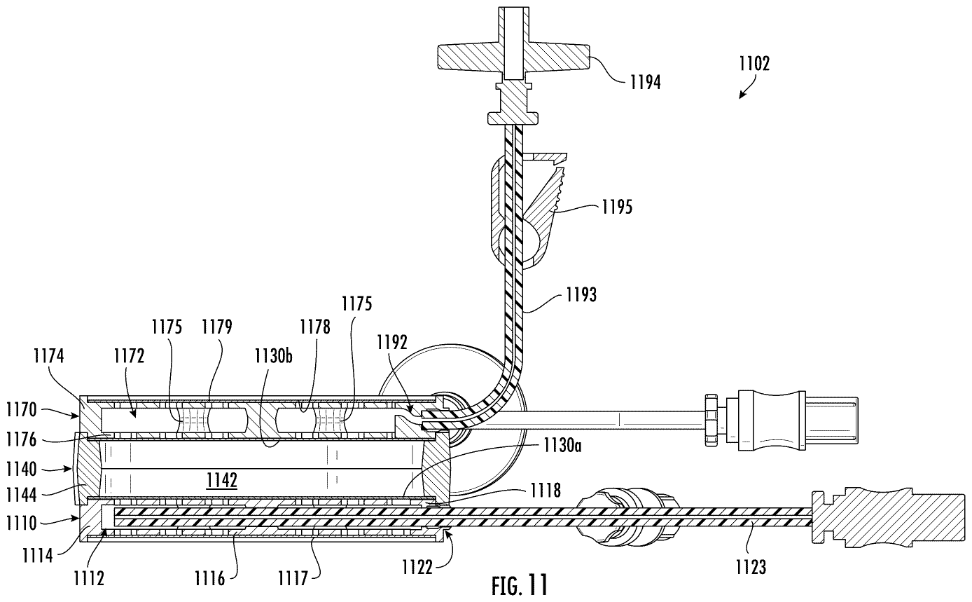

Referring now to FIGS. 10 and 11, another bioreactor 1102 is provided in accordance with embodiments of the present disclosure. The bioreactor 1102 is similar to the bioreactor 102 detailed above with like elements labeled with a similar label with a leading “11” replacing the leading “1” of the label of the similar element of the bioreactor 102 with only the differences detailed herein for brevity. The bioreactor 1102 may be used for static, perfusion, or recirculating processes.

The bioreactor 1102 is a three-chamber bioreactor including a first shell 1110, a second shell 1140, and a third shell 1170 that define a first chamber 1112, a second or growth chamber 1142, and a third chamber 1172, respectively. As shown, the first chamber 1112 and the third chamber 1172 have a thickness that is substantially equal to one another and significantly less than the thickness of the second chamber 1142. For example, the second chamber 1142 may have a thickness or height that is 1 to 100 times the thickness of the first chamber 1112 and the third chamber 1172, e.g., 2 times. The first chamber 1112 may have a thickness that is equal to, less than, or greater than a thickness of the third chamber 1172. The first chamber 1112 may be configured to be fed media through a fill port 1122. The thickness of the first chamber 1112 may be sized and dimensioned to allow the media to fill the first chamber 1112 before flowing through the divider 1118 into the second chamber 1142. In some embodiments, the tube 1123 secured to the fill port 1122 may extend a substantial length into the first chamber 1112 such that media entering the fill port 1122 is distributed within the first chamber 1112. In certain embodiments, the tube 1123 may be perforated within the first chamber 1112 such that the tube 1123 evenly distributes media within the first chamber 1112. The thickness of the first chamber 1112 may allow for distribution of the flow of the media to be substantially equal across the area defined by the divider 1118. The distribution of fluid may act as a fluidized bed to feed cells growing near the surface of the growth membrane. The thickness of the first chamber 1112 may vary based on a contemplated flow rate or pressure of the media provided to the first chamber 1112.

In embodiments, one or more of the first shell 1114, the second shell 1144, or the third shell 1174 may be an internal support. For example, as shown, the third shell 1174 includes internal supports 1175 that extend between the divider 1176 and the end wall 1178 to maintain a height or thickness of the third chamber 1172. The internal supports 1175 may be formed in a manner to minimize a reduction of volume of the third chamber 1172. In some embodiments, the internal supports 1175 may be configured to alter the flow within the third chamber 1172. For example, the internal supports 1175 may be configured to encourage turbulent flow to mix media within the third chamber 1172 and/or may be configured to direct flow of media through the third chamber 1172. In certain embodiments, the internal supports 1175 may be configured to direct gasses towards a vent port to remove the gasses from within the bioreactor 1102.

When the first chamber 1112 is filled with media, the media flows through the divider 1118, a growth membrane 1130a secured to the divider 1118, and into the second chamber 1142. When the second chamber 1142 is filled with media, the media flows through the divider 1176, and a growth membrane 130b secured to the divider 1176, into the third chamber 1172. When the third chamber 1172 is filled, the media may flow out of the drain port 1188 defined in the third chamber 1172.

The bioreactor 1102 may include a priming port 1192 that receives a priming tube 1193 that is in fluid communication with the third chamber 1172. The priming port 1192 may be defined in the sidewall 1174 of the third shell 1170. The priming tube 1193 may terminate in a priming filter 1194. The priming filter 1194 or priming tube 1193 may include a check valve to prevent fluid from backflowing into the third chamber 1172. The bioreactor 1102 may include a tube clamp 1195 disposed about the priming tube 1193 to selectively open and close a lumen defined by the priming tube 1193. The priming tube 1193 and tube clamp 1195 may be utilized during filling of the bioreactor 1102 with media to purge gasses from within the bioreactor 1102.

The bioreactor 1102 may include a vent port 1196 that receives a vent tube 1197 that is in fluid communication with the second chamber 1142. The vent port 1196 may be defined in the third sidewall 1174 of the third shell 1170. The vent tube 1197 may terminate in a vent filter 1198 that vents gasses from within the second chamber 1142, and thus the bioreactor 1102. The vent filter 1198 or the vent tube 1197 may include a check valve to prevent fluid from backflowing through the vent port 1196 into the third chamber 1172. While not explicitly shown, the vent port 1196 may extend through the third sidewall 1174 and the divider 1176 such that the vent port 1196 is in direct fluid communication with the second chamber 1142.

The bioreactor 1102 may include one or more accessory ports 1132 that each receive a respective accessory tube 1133. Each accessory tube 1133 may be in fluid communication with a respective chamber, e.g., the first chamber 1112, the second chamber 1142, or the third chamber 1172. The accessories ports 1132 may be defined in the first sidewall 1114 or the third sidewall 1174. The accessory ports 1132 may allow for additives to be fed into the bioreactor 1102. The accessory ports 1132 may allow for sampling of media or cells within the bioreactor 1102. As noted above, one or more of the accessory tubes 1133 may be in direct fluid communication with the second chamber 1142, as shown in FIG. 10A. Specifically, an accessory port 1132 may include an internal conduit 1132a that receives fluid from the accessory tube 1133 and passes through the divider 1176 such that the port 1132 is in direct fluid communication with the second chamber 1142. In such embodiments, one or more of the accessory tubes 1133 may be used to feed and/or harvest cells into or from the second chamber 1142.

The first shell 1114, the second shell 1144, and the third shell 1174 may each be of monolithic construction. For example, each shell may be formed of additive manufacturing techniques. Once each shell is formed, the first shell 1114, the second shell 1144, and the third shell 1174 may be bonded, welded, adhered, or otherwise joined to one another such that the chambers defined by the shells are substantially sealed. Before the first shell 1114, the second shell 1144, and the third shell 1174 are joined to one another, the growth membranes 1130a, 1130b are each secured to the respective divider 1118, 1176.

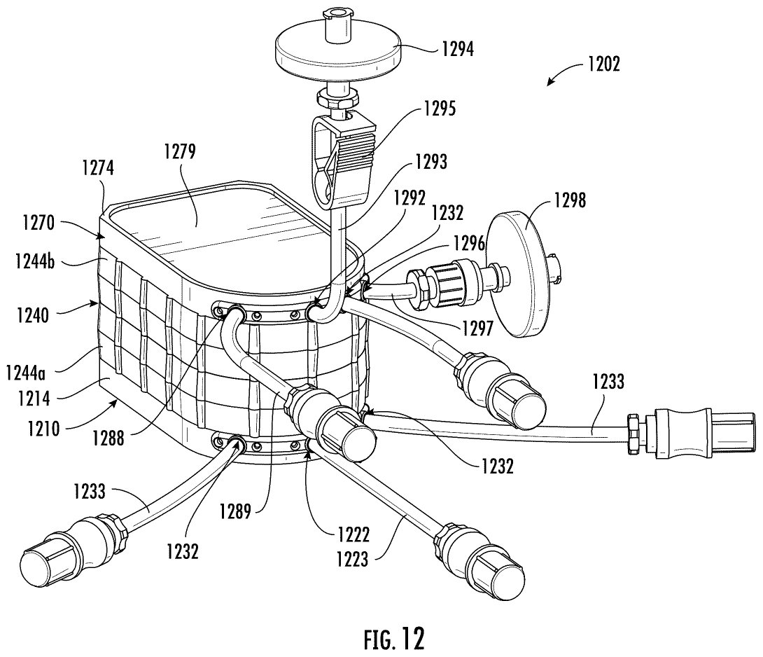

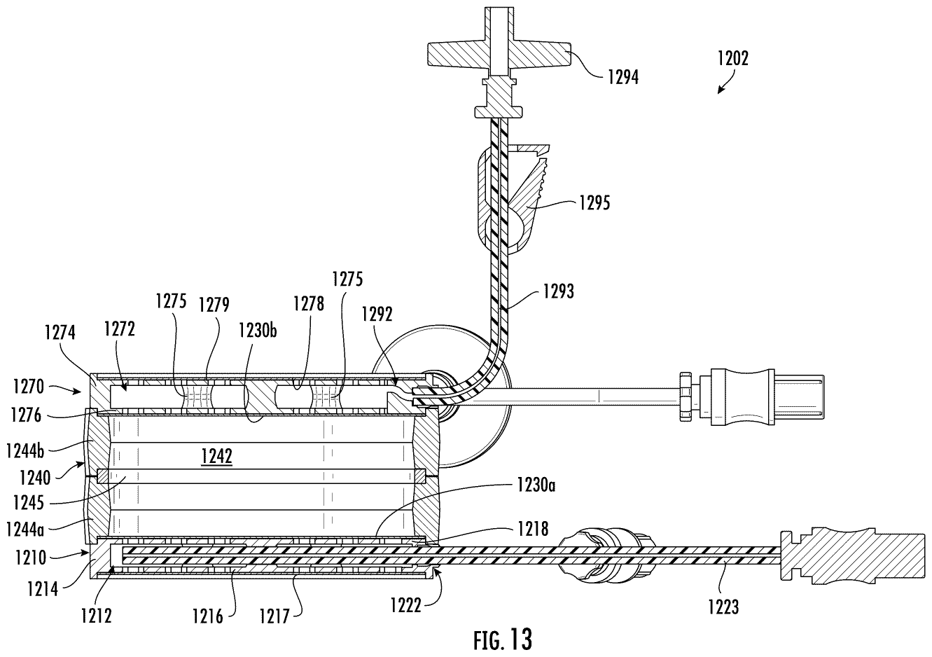

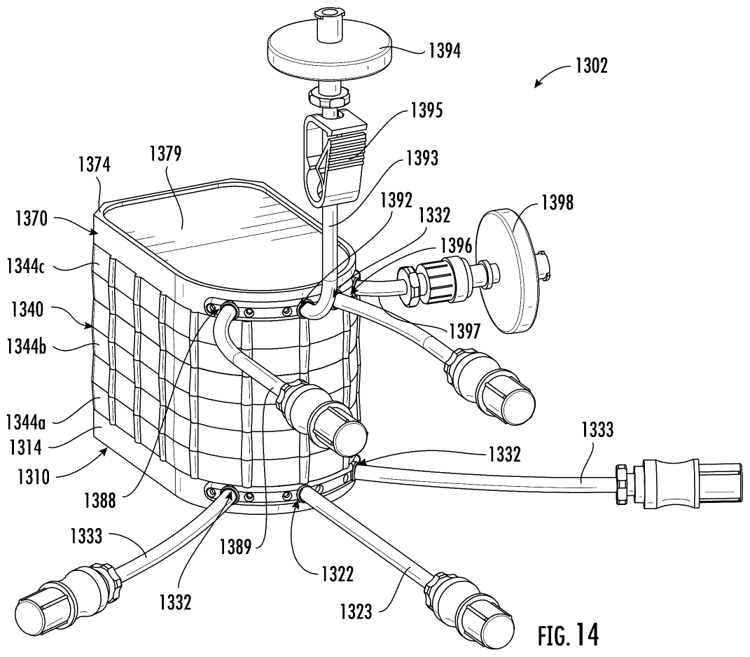

Referring now to FIGS. 12 and 13, another bioreactor 1202 is provided in accordance with embodiments of the present disclosure. The bioreactor 1202 is similar to the bioreactor 1102 detailed above with like elements labeled with a similar label with a leading “12” replacing the leading “11” of the label of the similar element of the bioreactor 1102 with only the differences detailed herein for brevity. The bioreactor 1202 may be used for static, perfusion, or recirculating processes.

The bioreactor 1202 is a three-chamber bioreactor including a first shell 1210, a second shell 1240, and a third shell 1270 that define a first chamber 1212, a second or growth chamber 1242, and a third chamber 1272, respectively. As shown, the first chamber 1212 and the third chamber 1272 have a thickness that is substantially equal to one another and significantly less than the thickness of the second chamber 1242. For example, the second chamber 1242 may have a thickness that is 1.5 to 6 times the thickness of the first chamber 1212 and the third chamber 1272, e.g., 4 times. The first chamber 1212 may have a thickness that is equal to, less than, or greater than a thickness of the third chamber 1272. The first chamber 1212 may be configured to be fed media through a fill port 1222. The thickness of the first chamber 1212 may be sized and dimensioned to allow the media to fill the first chamber 1212 before flowing through the divider 1218 into the second chamber 1242. The thickness of the first chamber 1212 may allow for distribution of the flow of the media to be substantially equal across the area defined by the divider 1218. The thickness of the first chamber 1212 may vary based on a contemplated flow rate or pressure of the media provided to the first chamber 1212.

The second chamber 1242 may be defined by one or more shells 1244. As shown, the second chamber 1242 is defined by second shells 1244a and 1244b. The shell 1244 may include a support band 1245 that extends between the shells 1244a, 1244b to support and seal a seam between the shells 1244a, 1244b. The increased volume of the second chamber 1242 may allow for an increased volume of cell growth within the second chamber 1242 during a single process.

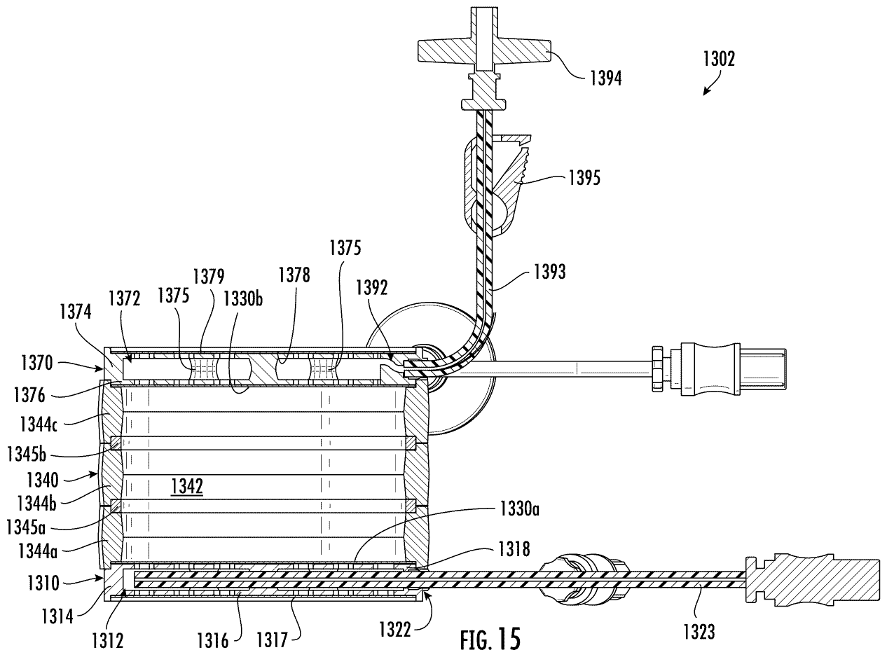

Referring now to FIGS. 14 and 15, another bioreactor 1302 is provided in accordance with embodiments of the present disclosure. The bioreactor 1302 is similar to the bioreactor 1202 detailed above with like elements labeled with a similar label with a leading “13” replacing the leading “12” of the label of the similar element of the bioreactor 1202 with only the differences detailed herein for brevity. The bioreactor 1302 may be used for static, perfusion, or recirculating processes. In some embodiments, the bioreactor is operated in static mode with the growth chamber partially filled with media and placed on a nuclear or orbital shaker to induce mixing.

The bioreactor 1302 is a three-chamber bioreactor including a first shell 1310, a second shell 1340, and a third shell 1370 that define a first chamber 1312, a second or growth chamber 1342, and a third chamber 1372, respectively. As shown, the first chamber 1312 and the third chamber 1372 have a thickness that is substantially equal to one another and significantly less than the thickness of the second chamber 1342. For example, the second chamber 1342 may have a thickness that is 1.5 to 9 times the thickness of the first chamber 1312 and the third chamber 1372, e.g., 6 times. The first chamber 1312 may have a thickness that is equal to, less than, or greater than a thickness of the third chamber 1372. The first chamber 1312 may be configured to be fed media through a fill port 1322. The thickness of the first chamber 1312 may be sized and dimensioned to allow the media to fill the first chamber 1312 before flowing through the divider 1318 into the second chamber 1342. The thickness of the first chamber 1312 may allow for distribution of the flow of the media to be substantially equal across the area defined by the divider 1318. The thickness of the first chamber 1312 may vary based on a contemplated flow rate or pressure of the media provided to the first chamber 1312.

The second chamber 1342 may be defined by one or more shells 1344. As shown, the second chamber 1342 is defined by second shells 1344a, 1344b, and 1344c. The shell 1344 may include support bands 1345a, 1345b that each extend between a pair of the shells 1344a, 1344b, 1344c to support and seal the respective seam between the pair of shells. The increased volume of the second chamber 1342 may allow for an increased volume of cell growth within the second chamber 1342 during a single process compared to the previously shown bioreactors.

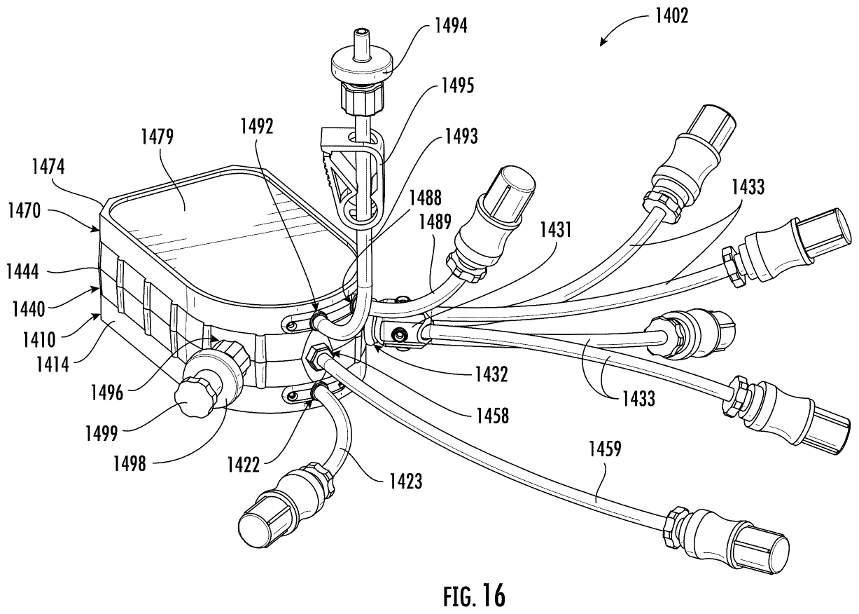

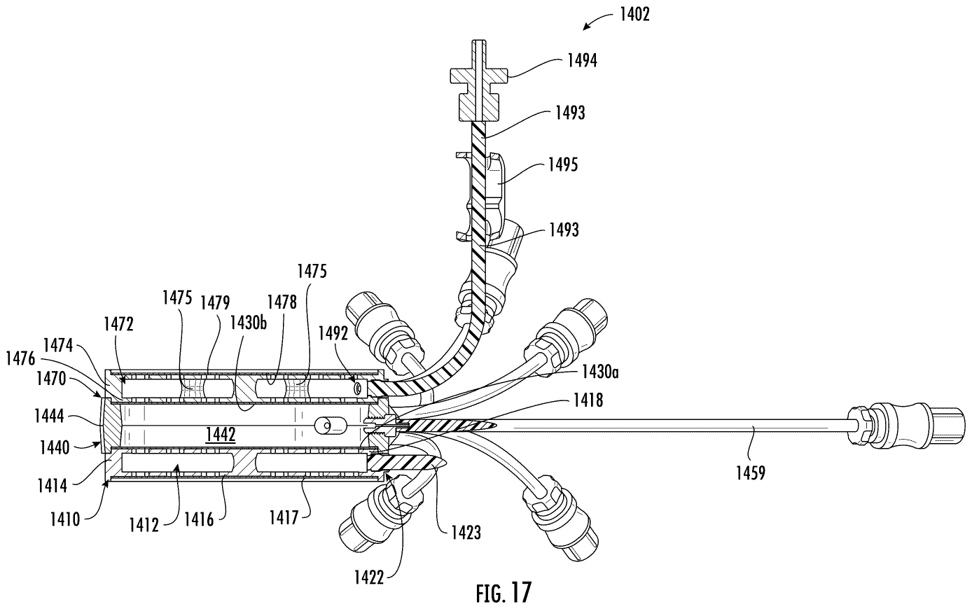

Referring now to FIGS. 16 and 17, another bioreactor 1402 is provided in accordance with embodiments of the present disclosure. The bioreactor 1402 is similar to the bioreactor 1102 detailed above with like elements labeled with a similar label with a leading “14” replacing the leading “11” of the label of the similar element of the bioreactor 1102 with only the differences detailed herein for brevity. The bioreactor 1402 may be used for static, perfusion, or recirculating processes.

The bioreactor 1402 is a three-chamber bioreactor including a first shell 1410, a second shell 1440, and a third shell 1470 that define a first chamber 1412, a second or growth chamber 1442, and a third chamber 1472, respectively. As shown, the first chamber 1412 and the third chamber 1472 have a thickness that is substantially equal to one another and significantly less than the thickness of the second chamber 1442. For example, the second chamber 1442 may have a thickness that is 1 to 10 times the thickness of the first chamber 1412 and the third chamber 1472, e.g., 2 times. The first chamber 1412 may have a thickness that is equal to, less than, or greater than a thickness of the third chamber 1472. The first chamber 1412 may be configured to be fed media through a fill port 1422. The thickness of the first chamber 1412 may be sized and dimensioned to allow the media to fill the first chamber 1412 before flowing through the divider 1418 into the second chamber 1442. In some embodiments, the tube 1423 secured to the fill port 1422 may extend a substantial length into the first chamber 1412 such that media entering the fill port 1422 is distributed within the first chamber 1412. In certain embodiments, the tube 1423 may be perforated within the first chamber 1412 such that the tube 1423 evenly distributes media within the first chamber 1412. The thickness of the first chamber 1412 may allow for distribution of the flow of the media to be substantially equal across the area defined by the divider 1418. The thickness of the first chamber 1412 may vary based on a contemplated flow rate or pressure of the media provided to the first chamber 1412.

The second shell 1444 defines ports 1432, 1458, and 1496. The accessory port 1432 may receive a multi-tube adaptor 1431 that places a plurality of lumens in direct fluid communication with the second chamber 1442. As shown, the multi-tube adapter 1431 splits a single lumen into four lumens to allow four accessory tubes 1433 to be in fluid communication with the second chamber 1442. As described above, the accessory tubes 1433 may allow for additives to be fed into the bioreactor 1402. The accessory port 1432 may allow for sampling of media or cells within the bioreactor 1402. One or more of the accessory tubes 1433 may be used to feed and/or harvest cells from the second chamber 1442. The port 1458 may be used as a fill or a drain port for the second chamber 1442. As such, the port 1458 may be used to feed and/or harvest cells from the second chamber 1442. The vent port 1496 may be configured to vent gasses from within the second chamber 1442. The vent port 1496 may include a check valve and/or a vent filter 1498 to allow gasses or liquid to escape the second chamber 1442 but prevent backflow of gasses or liquid into the second chamber 1442. The vent filter 1498 may prevent liquid from escaping the second chamber 1442. The vent filter 1498 may include a vent plug 1499 downstream of the vent filter 1498 to selectively open and close the vent port 1496. For example, the vent port 1496 may allow some gasses to escape the second chamber 1442 before the second chamber 1442 is filled with liquid but then be closed to prevent fluid from contacting the vent filter 1498 during the rest of the growing or expanding process.

While the bioreactor 1402 is shown with a single second shell 1444, the bioreactor 1402 may have additional second shells 1444 in a similar manner to the bioreactors 1202 and 1302. In addition, while the bioreactors 1102, 1202, and 1302 are shown with one, two, and three second shells, respectively, it should be appreciated that the bioreactors 1102, 1202, 1302, and 1402 are modular and can include as many second shells as desired for a respective growth process. In addition, the first and third shells of each of the bioreactors 1102, 1202, and 1302 may be the same such that additional second shells may be added without requiring additional components to be manufactured allowing for a reduction in the number of parts required to produce bioreactors having a different volume of the growth chamber.

The size of a second or growth chamber having a single second shell as described above may be in a range of 1 milliliters to 1000 milliliters, e.g., 25 milliliters. However, it is contemplated that the size of the bioreactors detailed above are scalable to accommodate large volumes. In view of the foregoing, the size of a second or growth chamber having a dual second shell may be in a range of 20 milliliters to 100 milliliters, e.g., 50 milliliters, and the size of a second or growth chamber having a triple second shell may be in a range of 30 milliliters to 150 milliliters, e.g., 75 milliliters.

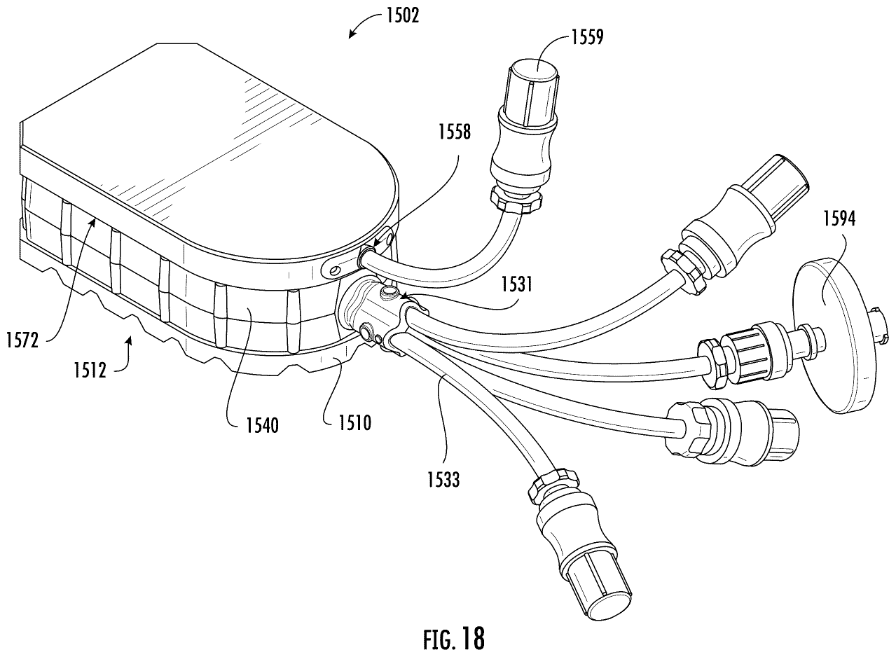

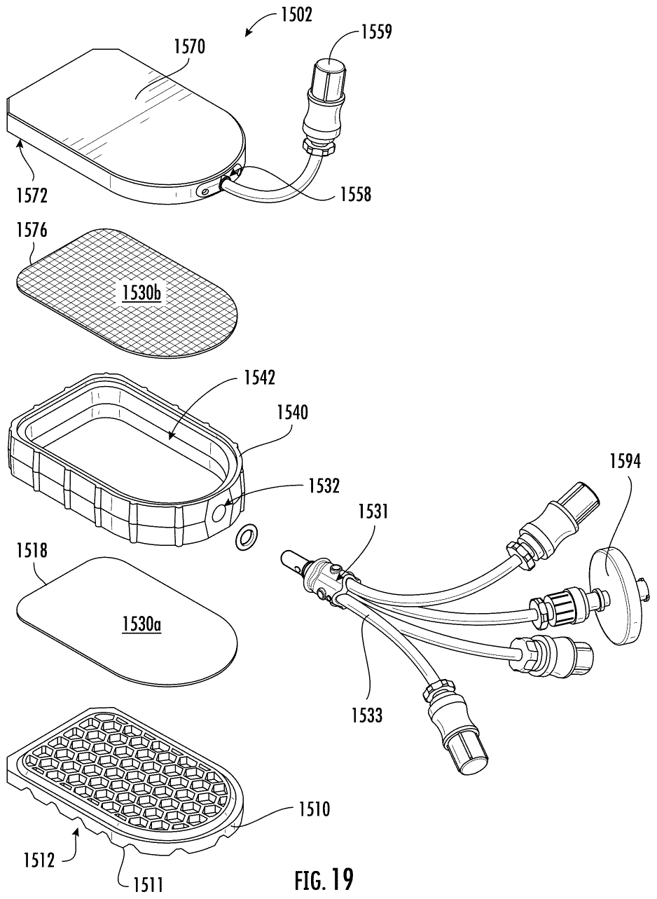

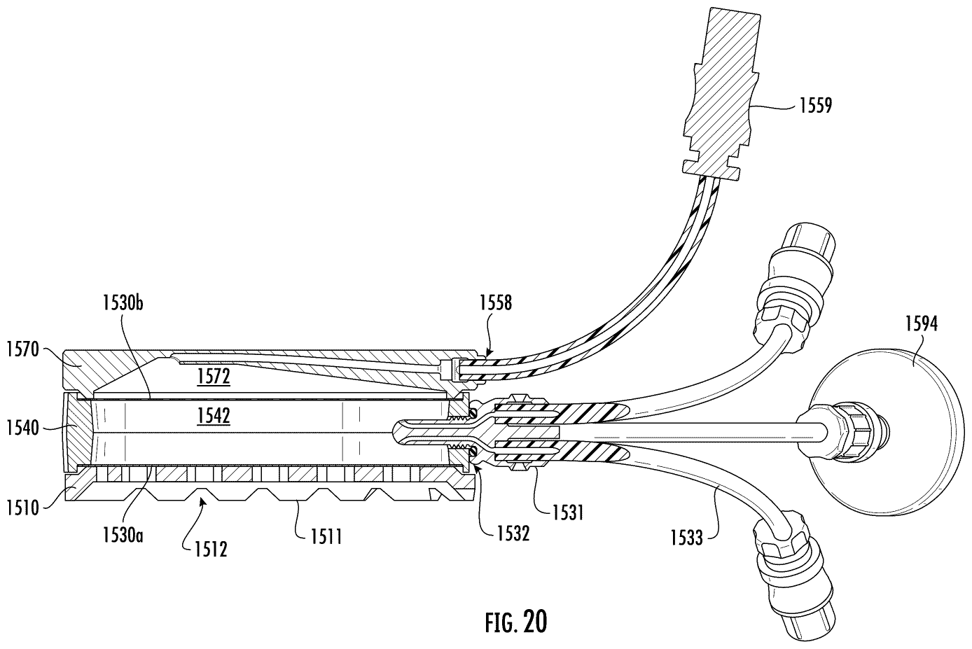

Referring now to FIGS. 18-20, another bioreactor 1502 is provided in accordance with embodiments of the present disclosure. The bioreactor 1502 is similar to the bioreactor 1102 detailed above with like elements labeled with a similar label with a leading “15” replacing the leading “11” of the label of the similar element of the bioreactor 1102 with only the differences detailed herein for brevity. The bioreactor 1502 may be used for static, perfusion, or recirculating processes.

The bioreactor 1502 is a two-chamber bioreactor including a first shell 1510, a second shell 1540, and a third shell 1570 that define a first chamber or space 1512, a second or growth chamber 1542, and a third chamber 1572, respectively. As shown, the space 1512 is positioned below the growth chamber 1542 and the bioreactor 1502 in general. The third chamber 1572 has a thickness that is significantly less than the thickness of the second chamber 1542. For example, the second chamber 1542 may have a thickness that is 1 to 10 times the thickness of the third chamber 1572, e.g., 2 times. The third chamber 1572 may be provided to collect and allow for the removal of spent media. The space 1512 may have a thickness that is equal to, less than, or greater than a thickness of the third chamber 1572. The thickness of the space 1512 may be sized and dimensioned to allow ambient air to contact a bottom surface of a divider 1518 before flowing through the divider 1518 into the second chamber 1542. The divider 1518 may be formed of a multilayer membrane 1530a. For example, the membrane 1530a may include a microporous track-etched membrane layer, a non-woven PET layer, and a silicone layer. The layers of the membrane 1530a may be in any order. In some embodiments, the silicone layer is directed towards the growth chamber 1542 and in other embodiments, the track-etched membrane layer is directed towards the growth chamber 1542. In some embodiments, the first shell 1510 includes a support lattice to support the underside of the membrane 1530a. In other embodiments, the membrane 1530a may only be supported by the edges of the first shell 1510 such that the center of the membrane 1530a, the portions away from the edges, is unsupported. The thickness of the space 1512 may vary based on a contemplated flow rate of air into and out of the space 1512. The edges of the first shell 1510 may define openings that allow for ambient air to flow into the space 1512. The edges 1511 of the first shell 1510 may be scalloped to define supports and openings spaced along the edges 1511 of the first shell 1510 to support the bioreactor 1502 on a support surface while allowing ambient air to flow into and out of the space 1512.

The second shell 1540 defines a single port 1532. The port 1532 may receive a multi-tube adaptor 1531 that places a plurality of lumens in direct fluid communication with the second chamber 1542. As shown, the multi-tube adapter 1531 splits a single lumen into four lumens to allow four accessory tubes 1533 to be in fluid communication with the second chamber 1542. As described above, the accessory tubes 1533 may allow for additives to be fed into the bioreactor 1502. The accessory port 1532 may allow for sampling of media or cells within the bioreactor 1502. One or more of the accessory tubes 1533 may be used to feed and/or harvest cells from the second chamber 1542. One of the accessory tubes 1533 may include a vent 1594 that is configured to vent gasses from within the second chamber 1542. The accessory tube 1533 including the vent 1594 may include a check valve and/or a vent filter to allow gasses or liquid to escape the second chamber 1542 but prevent backflow of gasses or liquid into the second chamber 1542. The vent filter may prevent liquid from escaping the second chamber 1542. The vent filter may include a vent plug downstream of the vent filter to selectively open and close the accessory tube 1533 including the vent 1594. For example, the vent 1594 may allow some gasses to escape the second chamber 1542 before the second chamber 1542 is filled with liquid but then be closed to prevent fluid from contacting the vent filter during the rest of the growing or expanding process.

The third shell 1570 is positioned above the second shell 1540 with a divider 1576 disposed between the growth chamber 1542 and the third chamber 1572 to separate the chambers 1542, 1572 from one another. The divider 1576 may be a membrane 1530b to separate the chambers 1542, 1572. The membrane 1530b may be a track-etched nonwoven backed membrane that allows media to pass through while preventing cells from passing through. The third shell defines a port 1558 may be used as a fill or a drain port for media into or out of the second chamber 1542. In certain embodiments, the port 1558 and the connector 1559 may be used to drain spent media from the second chamber 1542 via the third chamber 1572. The top of the third shell 1570 may be solid or may include a silicone layer. When the top of the third shell 1570 includes a silicone layer, the silicone layer may allow air to pass through while preventing media from passing through.

While the bioreactor 1502 is shown with a single second shell 1540, the bioreactor 1502 may have additional second shells 1540 in a similar manner to the bioreactors 1202 and 1302. In addition, while the bioreactors 1102, 1202, and 1302 are shown with one, two, and three second shells, respectively, it should be appreciated that the bioreactors 1102, 1202, 1302, and 1502 are modular and can include as many second shells as desired for a respective growth process. In addition, the first and third shells of each of the bioreactors 1102, 1202, and 1302 may be the same such that additional second shells may be added without requiring additional components to be manufactured allowing for a reduction in the number of parts required to produce bioreactors having a different volume of the growth chamber.

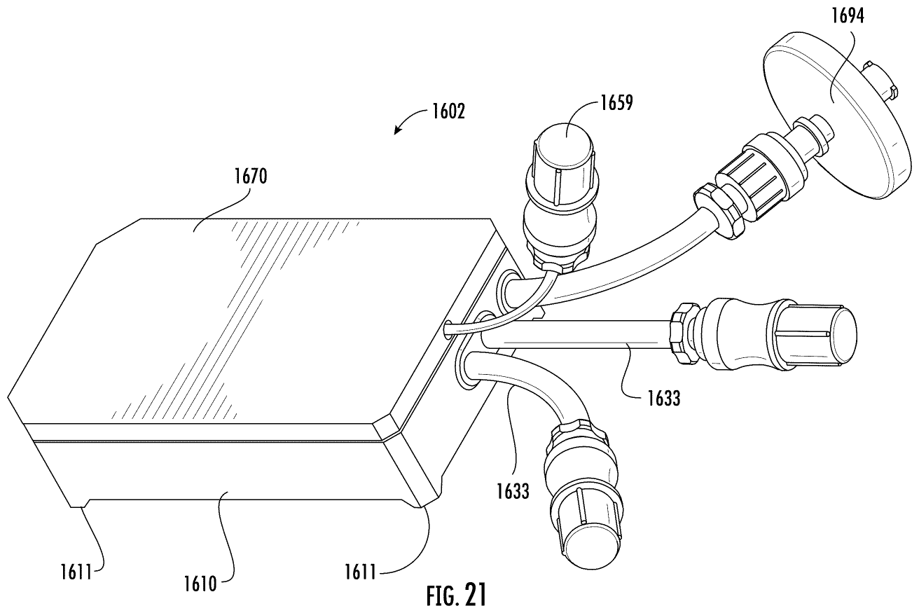

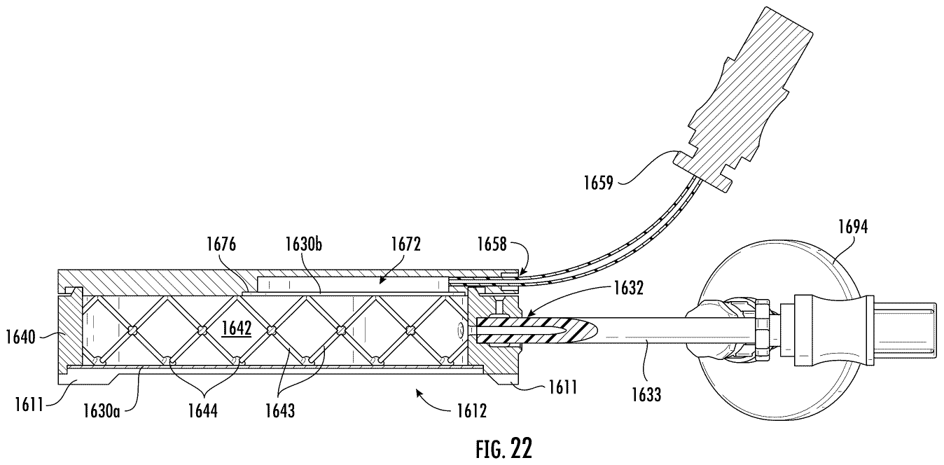

Referring now to FIGS. 21 and 22, another bioreactor 1602 is provided in accordance with embodiments of the present disclosure. The bioreactor 1602 is similar to the bioreactor 1102 detailed above with like elements labeled with a similar label with a leading “16” replacing the leading “11” of the label of the similar element of the bioreactor 1102 with only the differences detailed herein for brevity. The bioreactor 1602 may be used for static, perfusion, or recirculating processes.

The bioreactor 1602 is a two-chamber bioreactor including a first shell 1610, a second shell 1640, and a third shell 1670 that define a first chamber or space 1612, a second or growth chamber 1642, and a third chamber 1672, respectively. In some embodiments, the first shell 1610 and the second shell 1640 may be formed as a monolithic structure as shown in FIGS. 21 and 22. The space 1612 is positioned below the growth chamber 1642 and the bioreactor 1602 in general. The third chamber 1672 has a thickness that is significantly less than the thickness of the second chamber 1642. For example, the second chamber 1642 may have a thickness that is 1 to 10 times the thickness of the third chamber 1672, e.g., 2 times. The third chamber 1672 may be provided to collect and allow for the removal of spent media. The space 1612 may have a thickness that is equal to, less than, or greater than a thickness of the third chamber 1672. The thickness of the space 1612 may be sized and dimensioned to allow ambient air to contact a bottom surface of the membrane 1630a before flowing through the membrane 1630a into the second chamber 1642. The membrane 1630a may be a multilayer membrane. For example, the membrane 1630a may include a microporous track-etched membrane layer, a non-woven PET layer, and a silicone layer. The layers of the membrane 1630a may be in any order. In some embodiments, the silicone layer is directed towards the growth chamber 1642 and in other embodiments, the track-etched membrane layer is directed towards the growth chamber 1642. In some embodiments, the first shell 1610 includes a support lattice to support the underside of the membrane 1630a. In other embodiments, the membrane 1630a may only be supported by the edges of the first shell 1610 such that the center of the membrane 1630a, the portions away from the edges, is unsupported. The thickness of the space 1612 may vary based on a contemplated flow rate of air into and out of the space 1612. The edges of the first shell 1610 may define openings that allow for ambient air to flow into the space 1612. The edges of the first shell 1610 may be scalloped to define supports and openings spaced along the edges of the first shell 1610 to support the bioreactor 1602 on a support surface while allowing ambient air to flow into and out of the space 1612. In some embodiments, the first shell 1610 includes feet 1611 that space the membrane 1630a from a support surface to allow ambient air to flow into and out of the space 1612.

With particular reference to FIG. 22, the second chamber 1642 may include a lattice structure 1643 that extends within from the walls of the second chamber 1642. The lattice structure 1643 includes support pads 1644 formed along the interface with the membrane 1630a. The support pads 1644 engage with a top surface of the membrane 1630a to support the membrane 1630a. The lattice structure 1643 and the support pads 1644 may secure the membrane 1630a to the second shell 1642. In embodiments, the support pads 1644 may be adhered to the membrane 1630a. In some embodiments, the support pads 1644 are bonded to the membrane 1630a. For example, the support pads 1644 may be bonded by ultrasonic welding to the membrane 1630a. The lattice structure 1643 and the support pads 1644 may prevent or reduce sagging of the membrane 1630a. With the lattice structure 1643 and the support pads 1644 secured to the top surface of the membrane 1630a, the bottom surface of the membrane 1630a may be completely unsupported.

The second shell 1640 may define a single port 1632 or multiple ports 1632. The port 1632 may receive a multi-tube adaptor or multiple tubes to place one or more lumens in direct fluid communication with the second chamber 1642. As described above, the accessory tubes 1633 may allow for additives to be fed into the bioreactor 1602. The accessory port 1632 may allow for sampling of media or cells within the bioreactor 1602. One or more of the accessory tubes 1633 may be used to feed and/or harvest cells from the second chamber 1642. One of the accessory tubes 1633 may include a vent 1694 that is configured to vent gasses from within the second chamber 1642. The accessory tube 1633 including the vent 1694 may include a check valve and/or a vent filter to allow gasses or liquid to escape the second chamber 1642 but prevent backflow of gasses or liquid into the second chamber 1642. The vent filter may prevent liquid from escaping the second chamber 1642. The vent filter may include a vent plug downstream of the vent filter to selectively open and close the accessory tube 1633 including the vent 1694. For example, the vent 1694 may allow some gasses to escape the second chamber 1642 before the second chamber 1642 is filled with liquid but then be closed to prevent fluid from contacting the vent filter during the rest of the growing or expanding process.

The third shell 1670 is positioned above the second shell 1640 with a divider 1676 disposed between the growth chamber 1642 and the third chamber 1672 to separate the chambers 1642, 1672 from one another. The divider 1676 may be a membrane 1630b to separate the chambers 1642, 1672. The membrane 1630b may be a track-etched nonwoven backed membrane that allows media to pass through while preventing cells from passing through. The third shell defines a port 1658 may be used as a fill or a drain port for media into or out of the second chamber 1642. In certain embodiments, the port 1658 and the connector 1659 may be used to drain spent media from the second chamber 1642 via the third chamber 1672. The top of the third shell 1670 may be solid or may include a silicone layer. When the top of the third shell 1670 includes a silicone layer, the silicone layer may allow air to pass through while preventing media from passing through.

In some embodiments, the third chamber 1672 is directly accessible by one or more ports 1658 that allows for fluid communication with the third chamber 1672. The multiple ports 1658 may allow for additives or nutrients to be added directly to the third chamber 1672 and for spent media to be removed from the third chamber 1672. Adding nutrients and removing media from the third chamber may allow for perfusion of media entirely within the third chamber 1672 with the fresh media, e.g., nutrients, to flow into one of the ports 1658 and spent media to flow out of the other port 1658. The fresh media may flow through the membrane 1630b into the growth chamber 1642 and the spent media may flow from the growth chamber 1642 into the chamber 1672 through the membrane 1630b. It will be appreciated that the membrane 1630b retains cells within the growth chamber 1642.

While the bioreactor 1602 is shown with a single second shell 1640, the bioreactor 1602 may have additional second shells 1640 in a similar manner to the bioreactors 1202 and 1302. In addition, while the bioreactors 1102, 1202, and 1302 are shown with one, two, and three second shells, respectively, it should be appreciated that the bioreactors 1102, 1202, 1302, and 1602 are modular and can include as many second shells as desired for a respective growth process. In addition, the first and third shells of each of the bioreactors 1102, 1202, and 1302 may be the same such that additional second shells may be added without requiring additional components to be manufactured allowing for a reduction in the number of parts required to produce bioreactors having a different volume of the growth chamber.

The size of a second or growth chamber having a single second shell as described above may be in a range of 1 milliliters to 1000 milliliters or 10 milliliters to 100 milliliters, e.g., 25 milliliters. However, it is contemplated that the size of the bioreactors detailed above are scalable to accommodate large volumes. In view of the foregoing, the size of a second or growth chamber having a dual second shell may be in a range of 20 milliliters to 100 milliliters, e.g., 50 milliliters, and the size of a second or growth chamber having a triple second shell may be in a range of 30 milliliters to 150 milliliters, e.g., 75 milliliters.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

1. A bioreactor comprising:
a first shell including a first sidewall and a first end wall, the first sidewall defining a first chamber having a first end, the first end wall closing the first end;
a second shell including a second sidewall, the second sidewall defining a second chamber having second open end opposite the first end;
a first membrane secured across the second open end, the first membrane substantially sealing the second chamber at the second open end; and
a second membrane disposed between the first chamber and the second chamber, the second membrane being microporous and configured to allow liquid media to pass through while retaining cells within the second chamber.

2. The bioreactor according to claim 1, further comprising a lattice structure secured to the second shell, the lattice structure including pads, the first membrane secured to the pads.

3. The bioreactor according to claim 2, wherein the pads are adhered or bonded to the first membrane.

4. The bioreactor according to claim 1, wherein the second membrane is a track etched membrane that defines a plurality of pores, each pore having a diameter of less than or equal to 50 micrometers.

5. The bioreactor according to claim 1, wherein the second membrane is formed of a porous hydrophilic material.

6. The bioreactor according to claim 1, wherein the first membrane includes a semi-permeable layer of silicone, the semi-permeable layer of silicone configured to allow oxygen and carbon dioxide to permeate therethrough while preventing liquid from passing therethrough.

7. The bioreactor according to claim 6, wherein the first membrane includes a non-woven fabric layer.

8. The bioreactor according to claim 6, wherein the first membrane defines a plurality of pores, each pore having a diameter of less than or equal to 2 micrometers.

9. The bioreactor according to claim 1, wherein the second sidewall defines a first opening, the first opening configured to allow for aseptic addition of media.

10. The bioreactor according to claim 1, further comprising a third shell including a third sidewall, the third sidewall defining a space below the first membrane.

11. The bioreactor according to claim 1, wherein the bioreactor is configured to be used with a static, perfused, or recirculated growth media.

12. A bioreactor system comprising:
a bioreactor according to claim 1, the first sidewall defining an outlet and the second sidewall defining an inlet; and
a pump configured to flow liquid into the inlet in the second sidewall such that the liquid flows into the second chamber and into the first chamber before discharging through the outlet defined in the first sidewall.

13. The bioreactor system according to claim 12, wherein the pump is configured to recirculate liquid that flows from the outlet in the first sidewall back into the inlet defined in the second sidewall.

14. A bioreactor comprising:
a first sidewall defining a growth chamber disposed between a first end and a second end defined by the first sidewall;
a first membrane disposed over and substantially sealing the first end;
a second membrane disposed over the second end, the second membrane configured to retain cells within the growth chamber; and
a lattice structure secured to the first sidewall, the first membrane secured to the lattice structure.

15. The bioreactor according to claim 15, wherein the lattice structure is disposed within the growth chamber.

16. The bioreactor according to claim 15, wherein the lattice structure includes pads, a top surface of the first membrane secured to the pads of the lattice structure.

17. The bioreactor according to claim 15, wherein the first sidewall includes feet that extend below the first membrane to support the first membrane above a support surface.

18. The bioreactor according to claim 15, wherein the first sidewall defines a port therethrough, the port providing direct fluid communication into and out of the growth chamber.

19. The bioreactor according to claim 15, wherein the first membrane includes a top surface and a bottom surface opposite the top surface, the top surface of the first membrane exposed to the growth chamber, the bottom surface of the first membrane completely unsupported.

20. A method of expanding cells, the method comprising:
initiating flow of growth media into a bioreactor such that the growth media flows into a growth chamber of the bioreactor, the growth chamber defined by a first sidewall of the bioreactor, a first membrane that substantially seals a bottom of the growth chamber, and a second membrane that separates the growth chamber from a second chamber;
feeding cells to be expanded into the growth chamber; and
controlling flow of growth media into the growth chamber of the bioreactor after feeding cells into the growth chamber such that spent growth media flows through the second membrane into the second chamber before being discharged from the bioreactor after passing through the second chamber.

21-59. (canceled)
